(19) United States
(12) Patent Application Publication
(10) Pub. No.: US 2026/0034174 A1
(43) Pub. Date: Feb. 5, 2026

(54) METHODS AND CONSTRUCTS FOR GENE EDITING BY CODING SEQUENCE REPLACEMENT

(71) Applicant: BAR-ILAN UNIVERSITY, Ramat Gan (IL)

(72) Inventors: Ayal HENDEL, Kiryat Ono (IL); Daniel ALLEN, Giv'at Shmuel (IL); Bryan ITKOWITZ, Kiryat Gat (IL)

(21) Appl. No.: 19/117,481

(22) PCT Filed: Sep. 14, 2023
(86) PCT No.: PCT/IL2023/051004

§ 371 Date: Apr. 1, 2025

(51) Int. Cl. A61K 35/17; C12N 9/22; C12N 15/11; C12N 15/90

(52) U.S. Cl. CPC A61K 35/17; C12N 9/226; C12N 15/11; C12N 15/907; C12N 2310/20

(57) ABSTRACT

The present invention provides a system and a method for editing of an endogenous gene by replacing a gene portion sequence thereof comprised in a single exon with a transgene sequence, the system comprising at least one genome editing reagent designed for generating a double strand break (DSB) in a region spanning the gene portion sequence; and a replacement nucleic acid molecule comprising a coding sequence replacement (CDSR) construct designed for serving as a template for homology-directed repair (HDR) triggered by the DSB.

### FIELD OF THE INVENTION

The present disclosure is generally directed to gene editing. Specifically, the invention relates to gene editing by replacement of coding sequences.

### BACKGROUND OF THE INVENTION

Severe combined immunodeficiency (SCID) is a group of multiple rare monogenic disorders characterized by defects in both cellular and humoral adaptive immunity. Patients are born healthy and due to being extremely susceptible to infections, they present with recurrent infections early in life which if left untreated can be fatal. The Recombination-activating genes (RAG) RAG1 and RAG2 are tightly linked and have convergent transcriptional orientations on chromosome 11 separated by ˜12 kb. The RAG genes encode proteins that, when complexed together, commence the lymphoid-specific variable (V), diversity (D), and joining (J) gene [V(D)J]recombination process by catalysing DNA double-strand breaks (DSBs) at the recombination signal sequences (RSSs) which flank the V, D, and J gene segments. V(D)J recombination is a critical step in the maturation of T and B cells as it is responsible for the generation of a diverse repertoire of T- and B-cell receptors (TCR and BCR, respectively). Thus, patients with disease-causing variants in the RAG genes typically present with the complete absence or significant reduction of T and B cells and the T-B-NK+ immune phenotype. V(D)J recombination has three main mechanisms of regulation: 1) lineage specificity, namely BCR and TCR gene rearrangement occurs in B cells and T cells, respectively; 2) immunoglobulin heavy-chain rearrangement occurs before immunoglobulin light chain rearrangement; and 3) allelic exclusion, namely once a T or B cell rearranges its receptor locus, only one functional allele is expressed in that cell. In addition to these general mechanisms, the transcription of the RAG1 and RAG2 genes is regulated by numerous highly conserved, lineage-specific, cis-acting sequences surrounding their respective coding sequences (CDSs) which control the spatial genomic organization inside the locus. The RAG genes are expressed exclusively in the G₀/G₁ stage and display a tightly linked genomic organization with regulated expression during specific phases of T and B lymphocyte development. More specifically, two limited waves of RAG1 and RAG2 expression are necessary for Ig heavy and light chain rearrangements after which their expression is promptly terminated. This process is orchestrated by a plethora of transcriptions factors and machinery that complex together with cis-regulatory elements and promoter regions in the RAG1/2 locus. Together, they create a chromatin hub that acts as a super enhancer for the expression of the RAG genes. This formation, as well as the chromatin structure and 3D architecture, are crucial to ensuring that RAG1/2 are only expressed during the requisite developmental window. Overexpression or expression of the RAG genes outside of this precise window can result in genomic instability and lymphocyte malignancy through the formation of translocations and/or deletions in cancer-causing genes. Unsuccessful termination of RAG1 and RAG2 expression is also associated with atypical thymus development, an aberrant lymphatic system, and immunodeficiency.

Currently, the only definitive curable treatment for SCID patients is allogeneic hematopoietic stem cell transplantation (HSCT) from a human leukocyte antigen (HLA)-matched donor. However, finding an HLA-matched donor is rare, and the alternative treatment, haploidentical HSCT, reduces the survival rate from >80% with an HLA-matched donor to 60-70%. Although successful HSCT promotes lymphoid lineage development resulting in a long-term patient survival rate, it is accompanied by a high risk of graft-versus-host disease.

An ideal alternative to searching for an HLA-matched donor is to genetically edit the patient's own CD34⁺ hematopoietic stem and progenitor cells (HSPCs) ex vivo to be subsequently returned to the patient as an autologous HSCT. CD34⁺ HSPCs' marked ability to reconstitute the immune system from a small number of cells, make these cells an attractive platform for gene-therapy applications. To that end, transgene delivery via lentiviral (LV) or gammaretroviral (γRV) vectors for ex vivo editing of patients' CD34⁺ HSPCs was previously reported in gene-therapy clinical trials. However, genome-editing-based treatments using γRV of chronic granulomatous disease (CGD), Wiskott-Aldrich syndrome (WAS), SCID-X1, and adenosine deaminase deficiency (ADA)-SCID resulted in the activation of proto-oncogenes leading, in some patients, to a leukemic transformation. Although steps have been taken to improve the safety of these viral vectors, transgene integration into tumor-suppressor loci has been observed and incomplete phenotypic correction, toxicity, dysregulated hematopoiesis, and insertional mutagenesis related to the semi-random integration and constitutive expression of the transgene into the genome remain major safety concerns. Despite these concerns, which are particularly severe for highly controlled and regulated genes such as the RAG1/2, LV-based gene therapy for RAG1 is currently undergoing clinical trials.

Accordingly, there is a need for a safer approach, more accurately directing the correction to the relevant target and maintaining the strict endogenous spatiotemporal gene regulation and expression of the transgene, thereby reducing side-effects.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present invention relates to gene editing of endogenous genes by replacing the coding sequence or a portion thereof that is contained in a single exon with a transgene.

In some embodiments, there is provided a system for editing of an endogenous gene by replacing a gene portion sequence thereof comprised in a single exon with a transgene sequence, the system comprising:
at least one genome editing reagent designed for generating a double strand break (DSB) in a region spanning the gene portion sequence; and
a replacement nucleic acid molecule comprising a coding sequence replacement (CDSR) construct designed for serving as a template for homology-directed repair (HDR) triggered by the DSB, the CDSR construct comprising:
   a. a left homology arm (LHA) comprising a sequence essentially identical to a sequence directly upstream of the gene portion sequence;
   b. a right homology arm (RHA) comprising a sequence essentially identical to a sequence directly downstream of the gene portion sequence; and
   c. the transgene sequence comprising an edited sequence and positioned between the LHA and the RHA,
wherein following replacing the gene portion sequence, an edited gene is generated, encoding an edited gene product.

In some embodiments, the endogenous gene has a coding sequence (CDS) that is comprised in a single exon. In some embodiments, the endogenous gene comprises mutations in a single exon.

In some embodiments, the endogenous gene is a gene expressed in the hematopoietic system. In some embodiments, the endogenous gene is a gene expressed in the immune system.

In some embodiments, the endogenous gene is a RAG gene. In some embodiments, the endogenous gene is RAG1 or RAG2 gene. In some embodiments, the endogenous gene is a human gene.

In some embodiments, the gene portion sequence comprises at least 10%, 20%, 30%, 40%, 50%, 60%, 70%, 80%, 90%, or 95% of the CDS of the endogenous gene.

In some embodiments, the gene portion sequence has a length of about 1-10000 bp. In some embodiments, the gene portion sequence comprises only CDS.

In some embodiments, the at least one genome editing reagent is suitable for use with a genome editing system selected from zinc finger nucleases (ZFNs), transcription-activator like effector nucleases (TALEN), meganucleases, and the clustered regularly interspaced short palindromic repeats (CRISPR/Cas) system. In some embodiments, the genome editing system is a CRISPR/Cas9 system. In some embodiments, the at least one genome editing reagent comprises a guide RNA for defining the position of the DSB.

In some embodiments, the DSB is located within the gene portion sequence.

In some embodiments, the distance between the LHA and the DSB is longer than the distance between the RHA and the DSB and the LHA is longer than the RHA, or the distance between the RHA and the DSB is longer than the distance between the LHA and the DSB and the RHA is longer than the LHA. In some embodiments, the distance between the LHA and the DSB or the distance between the RHA and the DSB is less than about 10 bp. In some embodiments, the LHA and/or the RHA has a length of about 20-5000 bp.

In some embodiments, the edited sequence comprises a sequence having at least 60%, 70%, 80%, 90%, 95%, 96%, 97%, 98%, or 99% identity to a wild-type genomic sequence corresponding to the gene portion sequence. In some embodiments, the edited sequence comprises a CDS. In some embodiments, the edited sequence comprises a CDS encoding an amino acid sequence having at least 80%, 90%, 95%, 96%, 97%, 98%, or 99% identity to a wild-type amino acid sequence encoded by the wild-type genomic sequence. In some embodiments, the CDS of the edited sequence is not identical to the CDS of the wild-type genomic sequence, but encodes an amino acid sequence identical to the wild-type amino acid sequence.

In some embodiments, the transgene further comprises at least part of the 3′-UTR of the endogenous gene. In some embodiments, the transgene further comprises at least one element downstream of the CDS of the edited sequence, which is not present in the endogenous gene.

In some embodiments, the transgene further comprises a sequence comprising a knock-in (KI) reporter gene sequence encoding a KI reporter gene product. In some embodiments, the KI reporter gene product comprises a truncated cell-surface gene marker. In some embodiments, the transgene is designed such that the edited sequence and the KI reporter gene sequence form a single CDS which encodes the edited gene product including the reporter gene product, and the KI reporter gene product comprises a cleavable site designed to release the KI reporter gene product from the edited gene product. In some embodiments, the KI reporter gene product comprises a T2A self-cleaving peptide linked to a truncated cell-surface marker.

In some embodiments, the transgene has a length of about 10-4000 bp.

In some embodiments, the replacement nucleic acid molecule further comprises a delivery vector, for delivery of the CDSR construct into a cell. In some embodiments, the delivery vector is a recombinant adeno-associated virus serotype 6 (rAAV6).

In some embodiments, the system further comprises a knock-out (KO) nucleic acid molecule comprising a KO construct which comprises a KO reporter gene sequence encoding a KO reporter gene product, wherein the KO construct is designed for incorporating the KO reporter gene sequence into an integration region in the endogenous gene sequence, thereby preventing expression of an endogenous gene product, and the KO reporter gene is different from the KI reporter gene.

In some embodiments, the KO construct comprises a KO left homology arm (KO-LHA) comprising a sequence essentially identical to a sequence directly upstream of the integration region; a KO right homology arm (KO-RHA) comprising a sequence essentially identical to a sequence directly downstream of the integration region; and a KO reporter gene positioned between the KO-LHA and the KO-RHA, wherein following incorporating the KO reporter gene, the endogenous gene expresses the KO reporter gene product instead of the endogenous gene product.

In some embodiments, there is provided an ex vivo or in vitro method for editing of an endogenous gene by replacing a gene portion sequence thereof comprised in a single exon with a transgene sequence, the method comprising:
a. providing stem cells or progenitor cells suitable for gene targeting;
b. contacting the cells with at least one genome editing reagent thereby generating a double strand break (DSB) in a region spanning the gene portion sequence;
c. contacting the cells with a replacement nucleic acid molecule comprising a coding sequence replacement (CDSR) construct serving as a template for homology-directed repair (HDR) triggered by the DSB, the CDSR construct comprising:
   i. a left homology arm (LHA) comprising a sequence essentially identical to a sequence directly upstream of the gene portion sequence,
   ii. a right homology arm (RHA) comprising a sequence essentially identical to a sequence directly downstream of the gene portion sequence, and
   iii. the transgene sequence comprising an edited sequence and positioned between the LHA and the RHA; and
isolating edited cells comprising an edited gene encoding an edited gene product.

In some embodiments, contacting the cells with at least one genome editing reagent in step (b) is conducted by electroporation. In some embodiments, contacting the cells with at least one genome editing reagent in step (b) is conducted by electroporating the cells with a CRISPR/Cas9 RNP complex comprising Cas9 complexed with a single-guide RNA (sgRNA).

In some embodiments, contacting the cells in step (c) comprises transducing the cells with the replacement nucleic acid molecule at a multiplicity of infection (MOI) of less than 20000 viral genomes/cell, wherein the replacement nucleic acid molecule comprises a viral or retroviral vector sequence.

In some embodiments, the transgene further comprises a sequence comprising a knock-in (KI) reporter gene sequence encoding a KI reporter gene product.

In some embodiments, the KI reporter gene product is a truncated cell-surface marker.

In some embodiments, the transgene is designed such that the edited sequence and the KI reporter gene sequence form a single coding sequence (CDS) which encodes the edited gene product including the KI reporter gene product, and the KI reporter gene product comprises a cleavable site designed to release the KI reporter gene product from the edited gene product.

In some embodiments, the KI reporter gene product comprises a T2A self-cleaving peptide linked to a truncated cell-surface marker. In some embodiments, the KI reporter gene product comprises a T2A self-cleaving peptide followed by a truncated cell-surface marker.

In some embodiments, isolating the edited cells in step (d) comprises enriching for cells expressing the KI reporter gene product.

In some embodiments, the endogenous gene is not expressed in the stem cells or progenitor cells, and the isolating in step (d) further comprises growing the cells under differentiation conditions suitable for inducing expression of the endogenous gene and enriching for cells expressing the KI reporter gene product.

In some embodiments, the method further comprises at step (c) contacting the cells with a KO nucleic acid molecule comprising a KO construct which comprises a KO reporter gene encoding a KO reporter gene product, wherein the KO construct is designed for incorporating the KO reporter gene into an integration region in the endogenous gene sequence, thereby preventing expression of an endogenous gene product, and the KO reporter gene is different from the KI reporter gene.

In some embodiments, isolating the cells in step (d) comprises enriching for cells expressing both the KI reporter gene product and the KO reporting gene product.

In some embodiments, the stem cells or progenitor cells are hematopoietic or immune stem or progenitor cells. In some embodiments, the stem cells or progenitor cells are CD34⁺ hematopoietic stem and progenitor cells (HSPCs). In some embodiments, the stem cells or progenitor cells are pluripotential stem cells. In some embodiments, the stem cells or progenitor cells are derived from a subject suffering from a disease or disorder caused by a mutation in the endogenous gene. In some embodiments, the endogenous gene is the RAG1 gene or the RAG2 gene. In some embodiments, the stem cells or progenitor cells are derived from a healthy donor.

In some embodiments, there is provided an edited cell comprising an edited gene, obtained by the methods disclosed herein.

In some embodiments, there is provided a use of the system for editing of an endogenous gene disclosed herein, or of the edited cell comprising an edited gene disclosed herein, for treating a disease or disorder caused by a mutation in the endogenous gene in a subject in need thereof.

In some embodiments, there is provided the system for editing of an endogenous gene disclosed herein, or the edited cell comprising an edited gene disclosed herein, for use in treating a disease or disorder caused by a mutation in the endogenous gene in a subject in need thereof.

In some embodiments, there is provided a method of treating a disease or a disorder caused by a mutation in an endogenous gene in a subject in need thereof, comprising administering to the subject the edited cells comprising the edited gene obtained by the method disclosed herein.

In some embodiments, the cells are autologous cells of the subject.

In addition to the exemplary aspects and embodiments described above, further aspects and embodiments will become apparent by reference to the figures and by study of the following detailed descriptions.

### BRIEF DESCRIPTION OF DRAWINGS

The invention will now be described in relation to certain examples and embodiments with reference to the following illustrative figures.

FIGS. 1A-1H show various strategies for homology directed repair (HDR): Cut-Site Insertion vs. CDS Replacement and adjusting homology arms.

FIGS. 1A-1D—RAG2 gene. FIG. 1A shows a schematic representation of RAG2 KO disruption constructs containing a GFP reporter gene cassette under the control of an SFFV promoter and BGHpA 3′ sequence. Successful HDR of the CSI_GFP-BGHpA_400×400 constructs results in the integration of the reporter gene approximately 43 bp downstream from the RAG2 ATG start codon. Successful HDR of the three CDSR constructs results in the replacement of the entire endogenous RAG2 CDS with the reporter gene cassette. FIGS. 1B-1D show the results of HDR of the different constructs. Columns in each figure: A. CSI_GFP-BGHpA_400×400, B. CDSR_GFP-BGHpA_400×400, C. CDSR_GFP-BGHpA_400×800, D. CDSR_GFP-BGHpA_400×1600. Each pair (for FIGS. 1B-1C): left column control (rAAV6-only), right column test (CRISPR+AAV). FIG. 1B shows HDR frequencies analyzed by flow cytometry. Results: A (N=11, mean HDR 21.8%), B (N=10, Mean HDR 9.1%), C (N=11, Mean HDR 14.8%), D (N=14, 25.2%). rAAV6 only—mean HDR 1%. FIG. 1C shows site-specific HDR efficiencies at the RAG2 locus measured by droplet digital PCR (ddPCR) and normalized by targeted CCRL2 alleles. Results: A (rAAV6 only: N=4, mean HDR 0.3%; CRISPR+AAV: N=5, mean HDR 25.8%), B (rAAV6 only: N=4, mean HDR 0.5%; CRISPR+AAV: N=6, mean HDR 10.9%), C (rAAV6 only: N=3, mean HDR 0.4%; CRISPR+AAV: N=5, mean HDR 12.4%), D (rAAV6 only: N=9, mean HDR 1.4%; CRISPR+AAV: N=10, mean HDR 21.3%). FIG. 1D shows MFI values of HDR⁺ cells analyzed by flow cytometry. Results: A (N=11, mean MFI 0.8×10⁶), B (N=10, mean MFI 2.3×10⁶), C (N=11, mean MFI 2.5×10⁶), D (N=14, mean MFI 2.8×10⁶). Data are represented as mean±SEM. * p<0.05, ** p<0.01, *** p<0.001, and **** p<0.0001 (Mann-Whitney test).

FIGS. 1E-1H—RAG1 gene. FIG. 1E shows a schematic representation of RAG1 KO disruption donors containing a GFP reporter gene cassette under the control of an SFFV promoter and BGHpA sequence. Successful HDR of the RAG1_CSI_GFP-BGHpA_800×800 donor results in the integration of the reporter gene approximately 20 bp downstream from the RAG1 ATG start codon. Successful HDR of the three CDSR donors (RAG1_CDSR_GFP-BGHpA_800×800, RAG1_CDSR_GFP-BGHpA_800×1600, and RAG1_CDSR_GFP-BGHpA_800×2000) results in replacement of the entire endogenous RAG1 CDS with the reporter gene cassette. FIGS. 1F-1H show the results of HDR of the different constructs. Columns in each figure: A. RAG1_CSI_GFP-BGHpA_800×800, B. RAG1_CDSR_GFP-BGHpA_800×800, C. RAG1_CDSR_GFP-BGHpA_800×1600, and D. RAG1_CDSR_GFP-BGHpA_800×2000. Each pair (for FIGS. 1F-1G): left column control (rAAV6-only), right column test (CRISPR+AAV). FIG. 1F shows HDR frequencies analyzed by flow cytometry. Results: A (N=8), B (N=8), C (N=8), and D (N=8). FIG. 1G shows Site-specific HDR efficiencies at the RAG1 locus measured by ddPCR and normalized by targeted CCRL2 alleles. Results: A ([rAAV6 only: N=3; CRISPR+AAV: N=3]), B ([rAAV6 only: N=4; CRISPR+AAV: N=4]), C ([rAAV6 only: N=3; CRISPR+AAV: N=3]), and D ([rAAV6 only: N=4; CRISPR+AAV: N=4]). FIG. 1H shows MFI values of HDR⁺ cells analyzed by flow cytometry. Results: A (N=4), B (N=4), C (N=4), and D (N=4). Data are represented as mean±SEM. * p<0.05, ** p<0.01, *** p<0.001, and **** p<0.0001 (Mann-Whitney test).

FIGS. 2A-2D show the effect of synthetic polyA and/or cis-acting promoter response elements (PREs) on transgene expression. FIG. 2A shows a schematic representation of RAG2 KO disruption constructs containing a GFP reporter gene cassette under the control of an SFFV promoter to test the effect of different elements at the 3′ UTR. Top to bottom constructs: CDSR_GFP-NoBGHpA_400×1600, CDSR_GFP-BGHpA_400×1600 (same as in FIG. 1A), and CDSR_GFP-WPRE-BGHpA_400×1600. Successful HDR of the three constructs results in replacement of the entire endogenous RAG2 CDS with the reporter gene cassette. FIGS. 2B-2D show the results of HDR of the different constructs. Columns in each figure: A. CDSR_GFP-NoBGHpA_400×1600, B. CDSR_GFP-BGHpA_400×1600, C. CDSR_GFP-WPRE-BGHpA_400×1600. Each pair (for FIGS. 2B-2C): left column control (rAAV6-only), right column test (CRISPR+AAV). FIG. 2B shows HDR frequencies analyzed by flow cytometry. Results: A (N=9, mean HDR 14.3%, control mean HDR 1.0%), B (N=14, mean HDR 25.2%, control mean HDR 1.0%), and C (N=7, mean HDR 27.9%, control mean HDR 1.1%). FIG. 2C shows site-specific HDR efficiencies at the RAG2 locus measured by ddPCR and normalized by targeted CCRL2 alleles. Results: A (rAAV6 only: N=5, mean HDR 1.4%; CRISPR+AAV: N=6, mean HDR 10.7%), B (rAAV6 only: N=9, mean HDR 0.6%; CRISPR+AAV: N=10, mean HDR 21.3%), and C (rAAV6 only: N=7, mean HDR 0.8%; CRISPR+AAV: N=7, mean HDR 22.7%). FIG. 2D shows MFI values of HDR⁺ cells analyzed by flow cytometry. Results: A (N=9, mean MFI 0.3×10⁶), B (N=14, mean MFI 2.8×10⁶), and C (N=7, mean MFI 1.6×10⁶). Data are represented as mean±SEM. * p<0.05, ** p<0.01, *** p<0.001, and **** p<0.0001 (Mann-Whitney test), “ns”: not significant.

FIGS. 3A-3D show KI-KO simulation of functional gene correction of RAG2 in healthy donor (HD)-derived HSPCs using a double sorting strategy. FIG. 3A shows a schematic representation of RAG2 rAAV6 constructs for KI-KO biallelic correction simulation. Top to bottom: a RAG2 knock-out (KO) construct CDSR_GFP-BGHpA_400×800 containing a GFP reporter gene cassette under the control of an SFFV promoter and a 3′ BGHpA sequence (also shown in FIG. 1A); three RAG2 correction constructs CDSR_Corr_Endo3′UTR, CDSR_Corr_BGHpA, and CDSR_Corr_WPRE-BGHpA for knock-in of a dcoRAG2 cDNA sequence, comprising a T2A-tNGFR self-cleavable reporter gene to be expressed under the control of the endogenous RAG2 gene promoter, while the second and the third constructs further comprise a BGHpA 3′ element, or both a BGHpA and a WPRE 3′ elements, respectively. Successful HDR of the four constructs results in the replacement of the entire endogenous RAG2 CDS with the reporter gene cassette. FIG. 3B shows a two-step FACS enrichment approach for KI-KO multiplexed HDR gene-targeted CD34⁺ HSPCs post-CRISPR-Cas9/rAAV6 editing with a combination of CDSR_GFP-BGHpA_400×800 (KO) and either CDSR_Corr_Endo3′UTR, CDSR_Corr_BGHpA, or CDSR_Corr_WPRE-BGHpA (KI). Top: representative FACS plots of the populations two days post-editing (day 0). Left panel—sorting for GFP expression, right panel—sorting for tNGFR expression. All three correction groups express GFP yet do not express tNGFR since the RAG2 locus does not undergo transcription until later in the T cell differentiation process. Enrichment for GFP⁺ cells is conducted, and cells are seeded into an in vitro T cell differentiation (IVTD) system. Bottom: Representative FACS plots of the populations after 14 days in the IVTD (day 14). All three correction groups express tNGFR at various levels. Enrichment for tNGFR⁺ cells is conducted, producing a homogenous double-positive tNGFR⁺/GFP⁺ population indicative of KI-KO biallelic integration. These cells are seeded back into the IVTD system for another 14 days. Left panel: CDSR_Corr_Endo3′UTR; middle panel: CDSR_Corr_BGHpA; right panel: CDSR_Corr_WPRE-BGHpA. FIG. 3C shows site-specific multiplex HDR efficiencies at the RAG2 locus measured by ddPCR and normalized by targeted CCRL2 alleles. After extraction of genomic DNA, quantification of targeted alleles with KI and KO constructs was conducted individually and the sum of the two indicated 100% enrichment of KI-KO cells. Columns: A. CDSR_Corr_Endo3′UTR (KI), B. CDSR_Corr_Endo3′UTR (total), C. CDSR_Corr_BGHpA (KI), D. CDSR_Corr_BGHpA (total), E. CDSR_Corr_WPRE-BGHpA (KI), F. CDSR_Corr_WPRE-BGHpA (total), X. CDSR_GFP-BGHpA 400×800 (KO). Results: A+B (N=9, KI 51%, total 104%), C+D (N=5, KI 51%, total 112%), and E+F (N=10, KI 44%, total 101%). The respective % targeting for the KO (X) was 53%, 60%, and 57%. Data are represented as mean±SEM. FIG. 3D shows tNGFR MFI measurement indicating the level of transgenic RAG2 expression on days 14 and 28 of IVTD normalized to MFI of CDSR_Corr_WPRE-BGHpA. Since the dcoRAG2 cDNA and the tNGFR are connected by the T2A sequence resulting in a 1:1 ratio between transgenic RAG2 and tNGFR. Columns: A. CDSR_Corr_Endo3′UTR, B. CDSR_Corr_BGHpA, C. CDSR_Corr_WPRE-BGHpA. Results day 14 (left three columns): A (N=10, mean 0.5), B (N=6, mean 0.9), and C (N=11, mean 0.9); Results day 28 (right three columns): A (N=6, mean 0.5), B (N=5, mean 0.7), and C (N=6, mean 1.0). Data are represented as mean±SEM. * p<0.05, ** p<0.01, *** p<0.001, and **** p<0.0001 (Mann-Whitney test).

FIGS. 4A-4E show that IVTD of KI-KO correction simulation cells produces CD3⁺TCRγδ⁺ and CD3⁺TCRαβ⁺ T cells. FIGS. 4A-4E columns: A. control cells (electroporated only), B. CSI_Corr, C. CDSR_Corr_Endo3′UTR, D. CDSR_Corr_BGHpA, E. CDSR_Corr_WPRE-BGHpA. FIG. 4A shows qRT-PCR quantification of endogenous RAG2 gene expression in the RAG2 KI-KO cells (B-E) compared to control cells (A) on day 28 of IVTD. Expression fold change is plotted relative to control cells. ND: not detected. (N=3). Data are represented as mean±SEM. FIG. 4B shows qRT-PCR quantification of dcoRAG2 cDNA expression in the RAG2 KI-KO cells (B-E) compared to control cells (A) on day 28 of IVTD. Expression fold change is plotted relative to CDSR_Corr_WPRE-BGHpA (E) and samples with no expression detected are plotted as ND. (N=3). Results: control (not detected), B (0.33), C (0.64), D (0.67), and E (1.00). Data are represented as mean±SEM. * p<0.05, ** p<0.01, *** p<0.001, and **** p<0.0001 (Mann-Whitney test). FIG. 4C shows qRT-PCR quantification of total RAG2 expression in the RAG2 KI-KO cells compared to control cells on day 28 of IVTD. Expression fold change is plotted relative to mock. (N=3). Results: A (1.0), B (0.21), C (0.26), D (0.22), and E (0.56). Data are represented as mean±SEM. * p<0.05, ** p<0.01, *** p<0.001, and **** p<0.0001 (Mann-Whitney test). FIG. 4D shows a summary of CD3 expression by mock and KI-KO populations on day 28 of IVTD. Results: A (N=6, 6%), B (N=5, 11%), C (N=6, 22%), D (N=5, 19%), and E (N=6, 20%). Data are represented as mean±SEM. FIG. 4E shows the distribution into TCRαβ (upper part of columns, dark gray) and TCRγδ (lower part of columns, white) expressing CD3⁺ cells for control and KI-KO populations on day 28 of IVTD. Results: A (N=6, 77/23), B (N=5, 47/53), C (N=6), 37/63, D (N=5, 33/67), and E (N=6, 40/60). Data are represented as mean±SEM.

FIGS. 5A-5B show that expression of dcoRAG2 cDNA induces normal TCRαβ and TCRγδ repertoire development. FIGS. 5A-5B columns: A. control cells (electroporated only), B. CSI_Corr, C. CDSR_Corr_Endo3′UTR, D. CDSR_Corr_BGHpA, E. CDSR_Corr_WPRE-BGHpA. Left group: TRB, right group: TRG. FIG. 5A shows Simpson's 1-D diversity index of TRB and TRG repertoires on day 28 of control and KI-KO populations. Results TRB: A (N=3, 0.94), B (N=3, 0.98), C (N=3, 0.93), D (N=3, 0.96), and E (N=3, 0.98). Results TRG: A (N=3, 0.97), B (N=3, 0.94), C (N=4, 0.92), D (N=3, 0.95), and E (N=4, 0.93). Data are represented as mean±SEM. FIG. 5B shows Shannon's H diversity index of TRB and TRG repertoires on day 28 of control and KI-KO populations. Results TRB: A (N=3, 4.40), B (N=3, 4.66), C (N=3, 3.93), D (N=3, 4.38), and E (N=3, 5.20). Results TRG: A (N=3, 4.37), B (N=3, 4.41), C (N=4, 3.96), D (N=3, 4.60), and E (N=4, 4.44). Data are represented as mean±SEM.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

In view of various unresolved issues of existing gene correction methods for treating severe combined immunodeficiency syndrome (SCID) caused by mutations in the recombination activation genes (RAG) genes, the present invention provides methods for a more precise replacement of a defective RAG gene, with minimal disturbance to the natural arrangement of the coding sequence with respect to genomic regulatory elements.

Previous attempts to correct the RAG genes lacked the ability to maintain endogenous regulatory and spatiotemporal elements since the integration of the transgene was either semi-random or via insertion of thousands of bp to the Cas9-induced cut site, thus distancing the RAG genes from one another and potentially altering the genomic locus and hindering optimal gene expression. Iancu et al. (Multiplex HDR for disease and correction modelling of SCID by CRISPR genome editing in human HSPCs, Mol Ther Nucleic Acids. 31:105-121) proposed a construct, referred to below as cut-site insertion (CSI)_Corr. However, this approach had two important shortcomings:

First, the non-diverged 3′ UTR sequence of the CSI_Corr construct could act as a 3′ homology arm to the identical endogenous 3′ UTR sequence and lead to incomplete or early cessation of homology-directed repair (HDR). Indeed, such events were identified by ONT long-read sequencing analysis following use of this construct.

Second, the incorporation of a complete reporter cassette in the CSI_Corr construct can have major implications on local chromatin structure and regulation. Chiefly concerning is the presence of a constitutive phosphoglycerate kinase (PGK) promoter. The insertion of such an element in a genomic locus like RAG1/2 that requires such tight regulation is a risk that should be eliminated.

Gardner et al. (Gene Editing Rescues In vitro T Cell Development of RAG2-Deficient Induced Pluripotent Stem Cells in an Artificial Thymic Organoid System, J Clin Immunol. 41(5):852-862) and Pavel-Dinu et al. (Genetically Corrected RAG2-SCID Human Hematopoietic Stem Cells Restore V(D)J-Recombinase and Rescue Lymphoid Deficiency, bioRxiv. 2022.07.12.499831) avoided this issue by introducing a BGHpA or WPRE-BGHpA sequence in place of the 3′ UTR. However, this solution did not overcome the second issue.

The present invention proposes to resolve both issues by replacing the coding sequence of the gene, or a part thereof, while affecting regulatory elements, either intronic or surrounding the gene, as little as possible.

As an added measure for safety and accuracy, while the RAG2 transgene (dcoRAG2) encodes a protein identical to wild-type RAG2, it introduces wobble changes in the amino acid codons, which lead to reduced similarity to the genomic sequence, thereby precluding the Cas9 from re-cutting the inserted sequence or from the inserted sequence serving as a homology arm causing premature cessation of HDR.

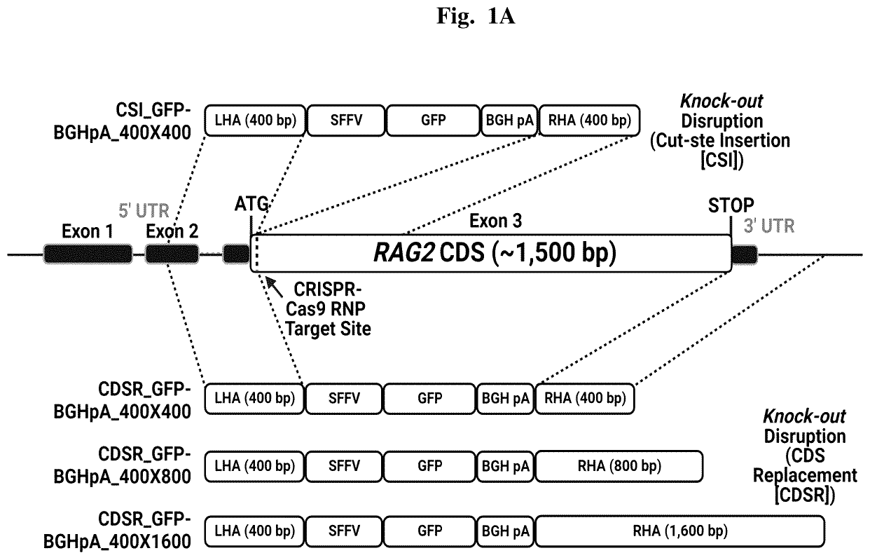

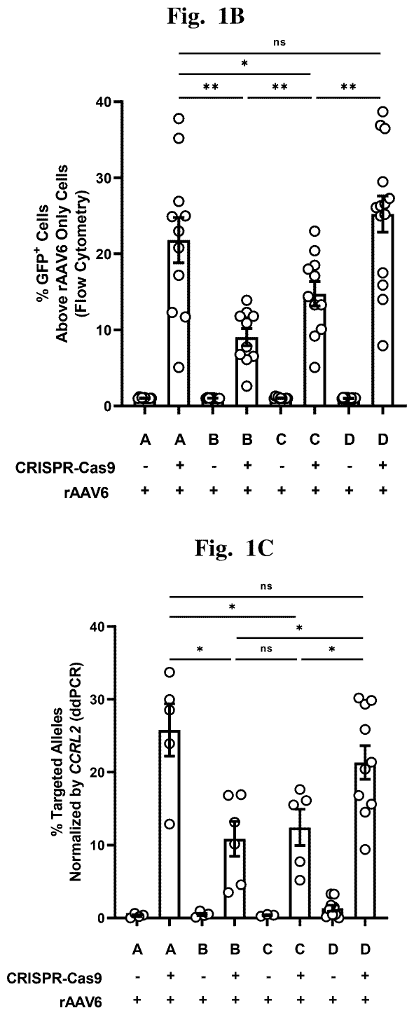

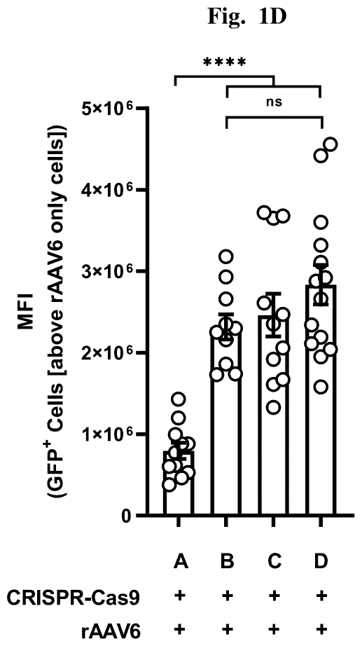

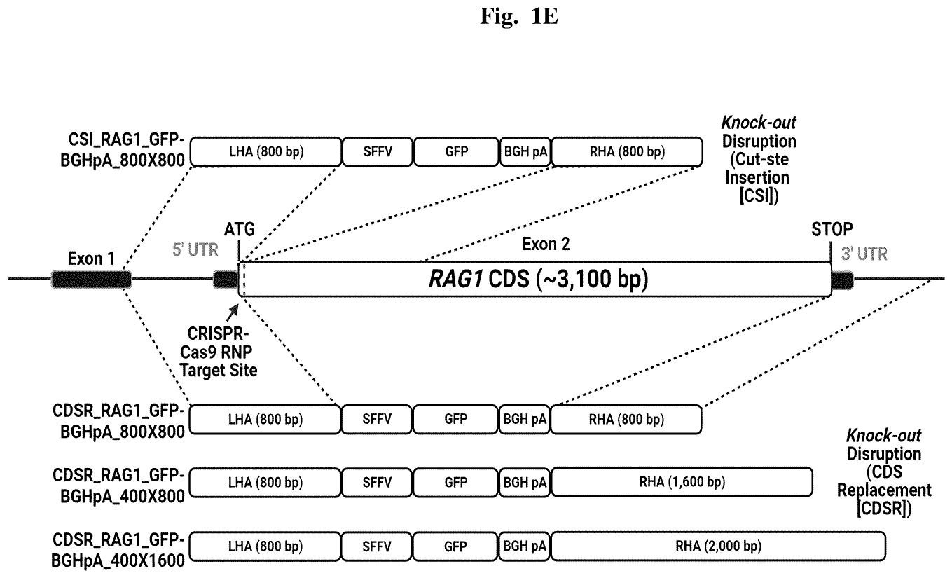

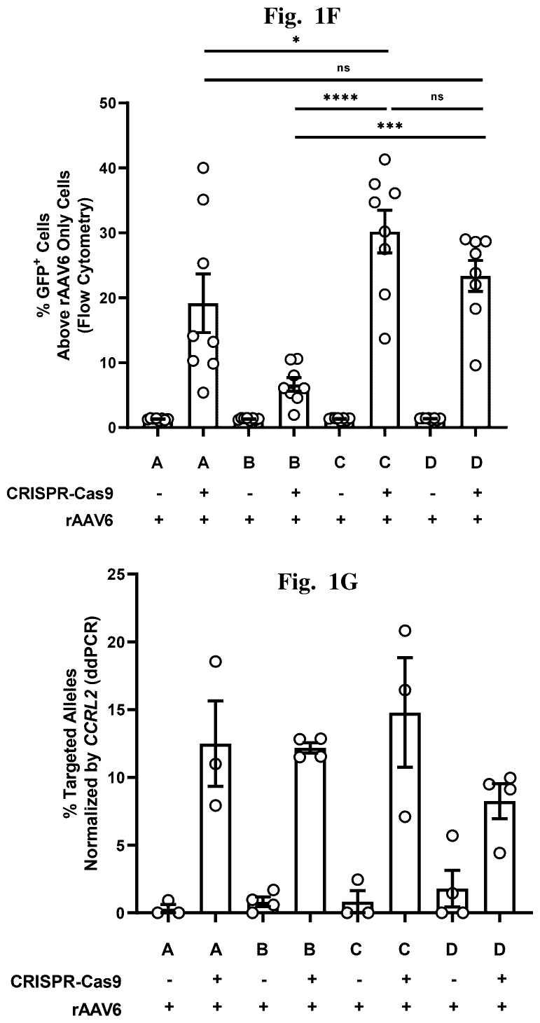

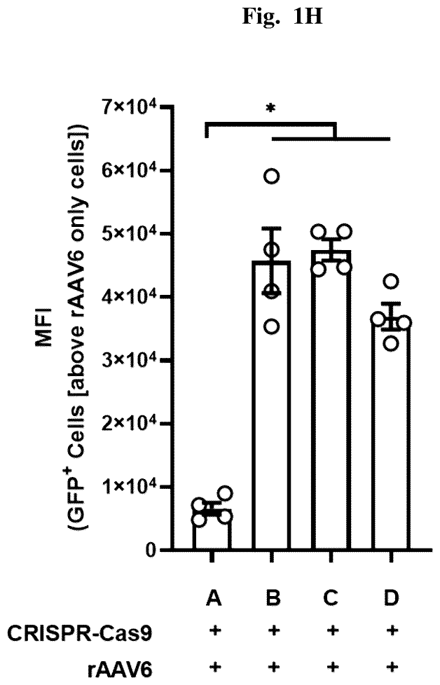

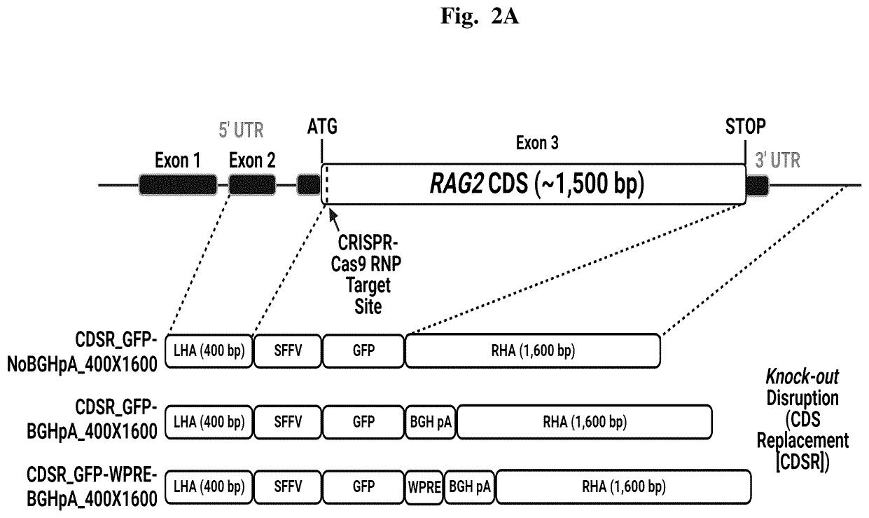

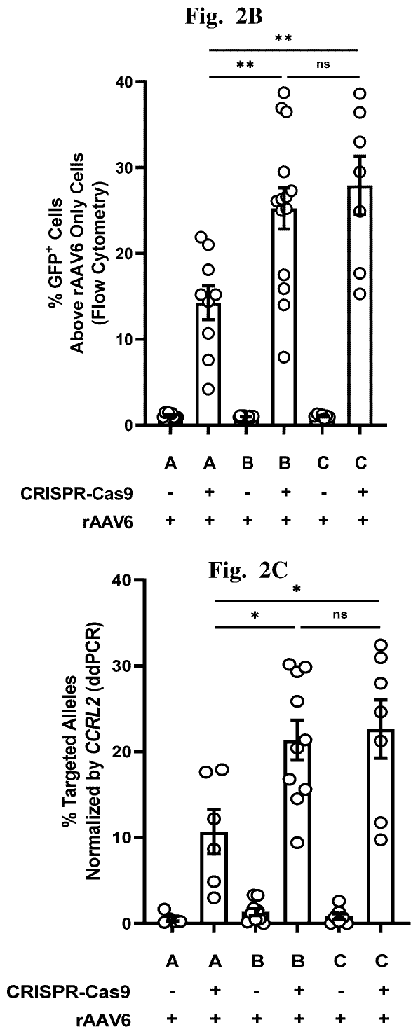

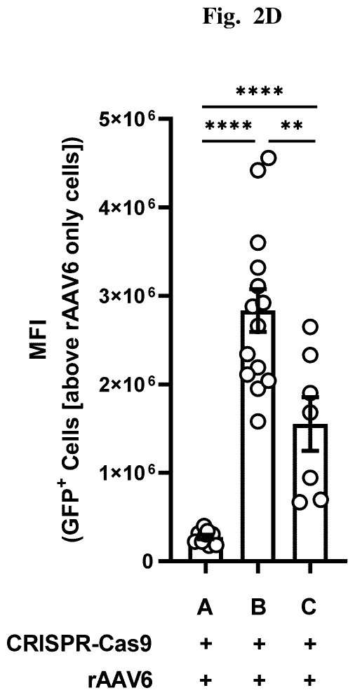

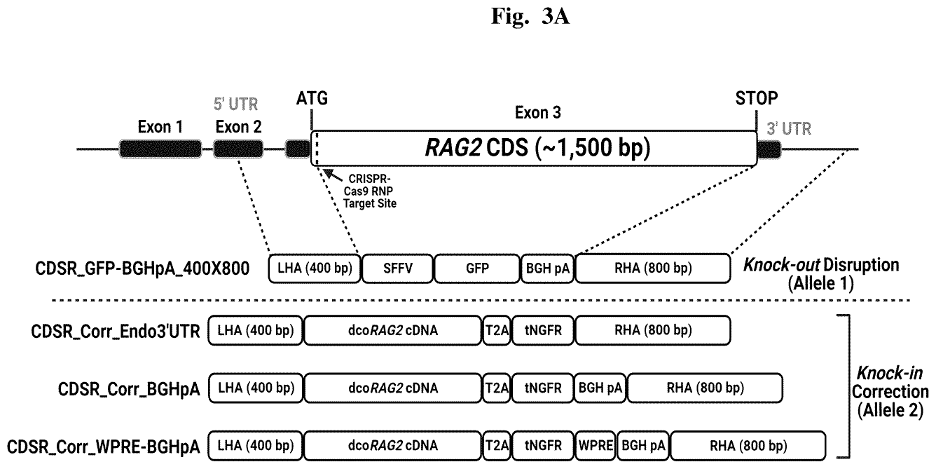

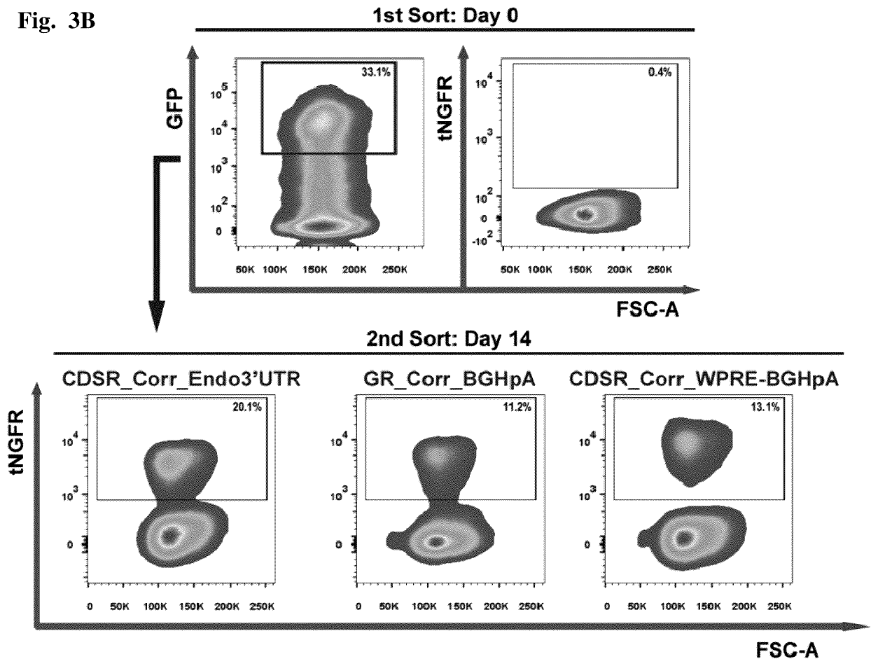

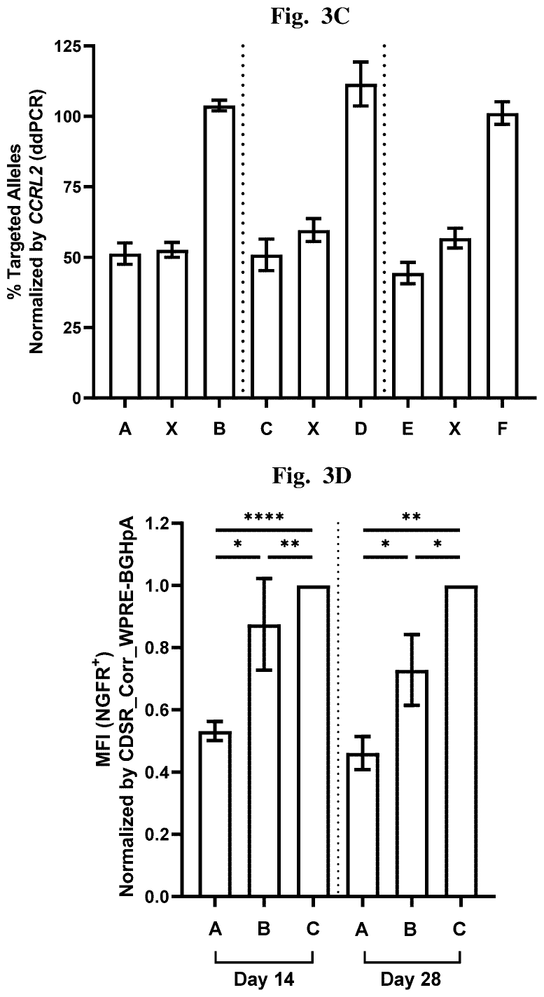

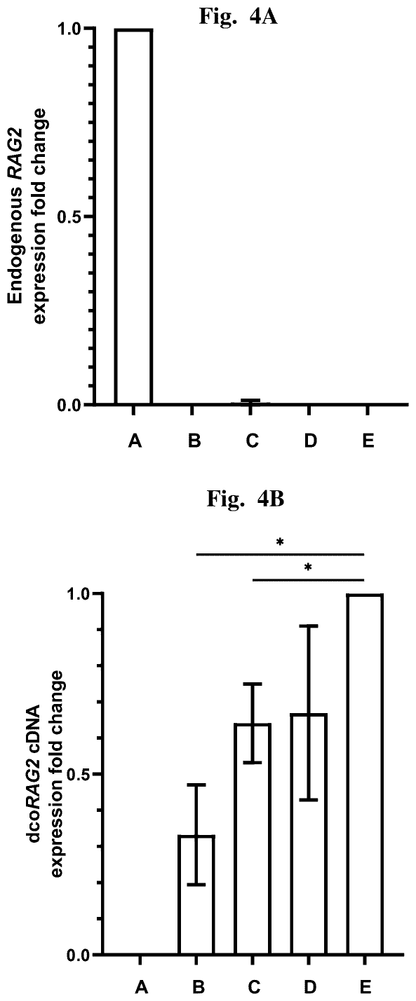

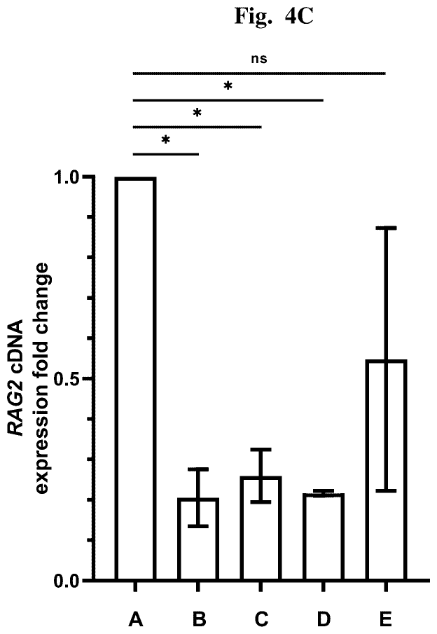

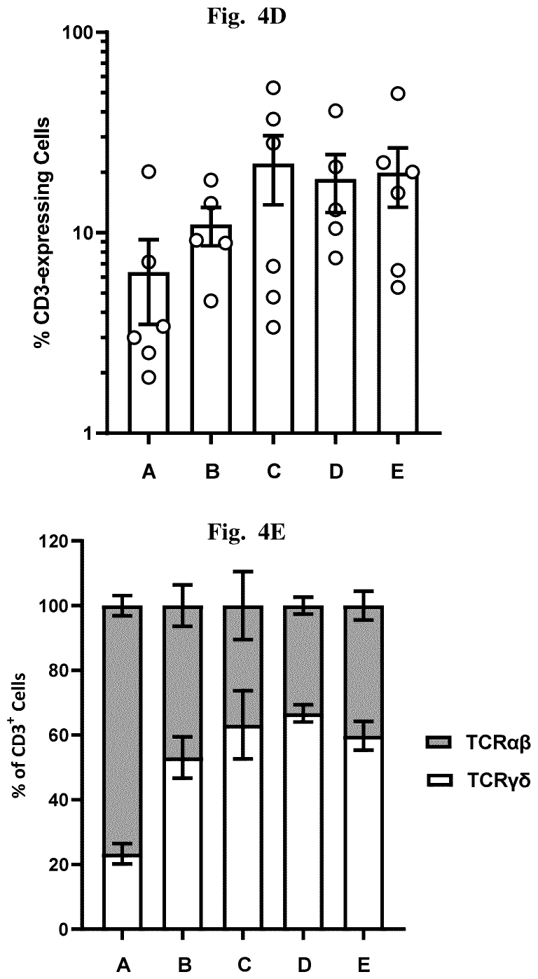

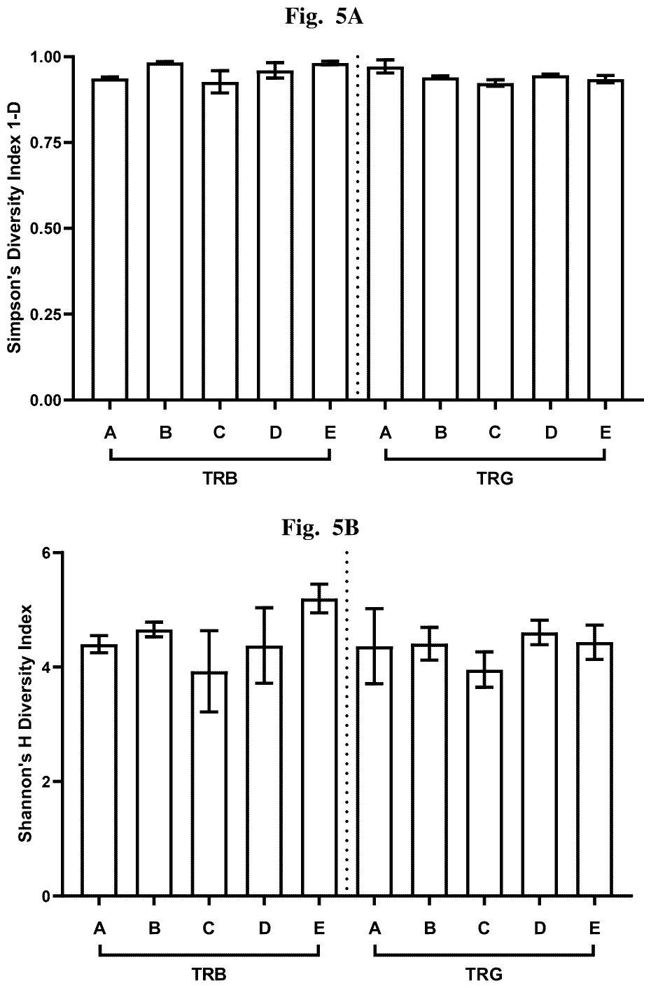

The constructs generated according to the present invention, termed coding sequence replacement (CDSR) constructs, were able to correctly integrate and replace the endogenous RAG2 coding sequence (CDS), as shown in FIGS. 1B-1D for knock-out (KO) constructs including various sizes of a right homology arm (RHA), and in FIGS. 2B-2D for KO constructs including various 3′-UTRs, including bovine growth hormone (BGH) polyA signal (BGHpA) and woodchuck hepatitis virus posttranscriptional regulatory element (WPRE) elements. FIGS. 3B-3D show a selection scheme for cells including both knock-in (KI) constructs and KO constructs, and correct integration and expression of the RAG2 transgene of the KI constructs. FIGS. 4A and 4B show the successful replacement of the endogenous RAG2 gene by the RAG2 transgene, and FIG. 4C shows that the naturally occurring RAG2 expression levels (of the control) is not exceeded in the transgenes, which present with about 25-50% of the control expression level. This is important since overexpression may cause genomic instability and lymphocyte malignancy. Further, as shown in FIGS. 4 and 5, appropriate cell differentiation and V(D)J recombination was achieved in spite of the expression being lower than the endogenous RAG2 expression. As shown in FIG. 4B, higher transgenic RAG2 mRNA levels were observed for the CDSR constructs than for the insertion (CSI) construct, highlighting the advantage of the CDSR strategy. FIG. 4B further shows a significantly higher level of transgenic RAG2 mRNA when the transgene expression was under the 3′ regulation of the WPRE-BGHpA sequence compared to the expression level under the endogenous 3′ UTR. Finally, edited (transgenic) T cells developed in vitro showed similar expression levels of T cell markers compared with the control (FIGS. 4D, 4E), as well as differentiation into CD3⁺TCRαβ⁺ and CD3⁺TCRγδ⁺ T cells, and a normal rate of V(D)J recombination (FIGS. 5A-5B), indicating adequate functional development of the transgenic T cells.

It is noted that while data is only presented for the two RAG genes it is believed that this replacement method may be applied to other genes having a single coding exon, or for exon replacement where known mutations are localized on a single exon, especially in monogenic diseases of the blood and immune system.

In some embodiments, there is provided a system for editing of an endogenous gene by replacing a gene portion sequence thereof comprised in a single exon with a transgene sequence, the system comprising:
at least one genome editing reagent designed for generating a double strand break (DSB) in a region spanning the gene portion sequence; and
a replacement nucleic acid molecule comprising a CDSR construct designed for serving as a template for HDR triggered by the DSB, the CDSR construct comprising:
   a. a left homology arm (LHA) comprising a sequence essentially identical to a sequence directly upstream of the gene portion sequence;
   b. a right homology arm (RHA) comprising a sequence essentially identical to a sequence directly downstream of the gene portion sequence; and
   c. the transgene sequence comprising an edited sequence and positioned between the LHA and the RHA,
   wherein following replacing the gene portion sequence, an edited gene is generated, encoding an edited gene product.

The phrase “editing an endogenous gene” refers to making a change in the endogenous gene inside a cell. The purpose of the editing may be for correcting a defect in the gene, such as correcting a mutation which may cause a disease. Other reasons for editing an endogenous gene may be for making a desired change in the gene.

The term “endogenous gene” relates to a genomic sequence defining a gene that is naturally present in a cell, as opposed to a transgene, which is an exogenous sequence inserted into the cell. Throughout the application, the term “gene” without a further identifier relates to the endogenous gene.

The term “edited gene” refers to a genomic sequence defining the endogenous gene after genomic editing by the system of the invention, in which the gene portion sequence, which is comprised in a single exon, has been replaced by the transgene sequence. In some embodiments, endogenous gene sequence not included in the gene portion sequence remains unchanged in the edited gene.

The term “edited gene product” relates to a gene product expressed from the edited gene, which includes the transgene sequence. In some embodiments, the edited gene product is an amino acid sequence, or a protein, which is encoded by or expressed from the edited gene.

The endogenous gene may be any gene for which editing is desired. Since the hematopoietic system can be relatively easily replaced or modified, e.g., by blood transfusion or by bone marrow transplantation, in some embodiments, the endogenous gene is a gene expressed in the hematopoietic system. In some embodiments, the endogenous gene is a gene expressed in the immune system.

The uniqueness of the system and method of the invention, as also explained above, is partly in that it edits a gene by replacing a part of the gene sequence (a gene portion sequence thereof) while affecting the gene regulation, which is crucial for the expression of the edited gene, as little as possible. For that purpose, the system of the invention is designed to replace only an exon or part thereof, but not introns, which may contain regulatory elements.

In some embodiments, the endogenous gene is comprised in a single exon. In some embodiments, the endogenous gene has a coding sequence which is comprised in a single exon. It is appreciated that the gene may comprise more than one exon, or the CDS may comprise more than one exon, but the sequence that is replaced (the gene portion sequence) is comprised in a single exon. For example, where there is a single mutation in the gene, and only the exon including the mutation is replaced by the system of the invention, or when all mutations are comprised in a single exon. The term “mutation” means a variation in the gene sequence from the normal sequence, which has functional adverse effects on the function of the gene.

The recombination-activating genes (RAG) include the genes RAG1 and RAG2, which play important roles in the rearrangement and recombination of the genes encoding immunoglobulin and T cell receptor molecules. These genes are expressed in lymphocytes during differentiation, and their expression is essential to the generation of mature B cells and T cells. Both RAG1 and RAG2 contain one protein-coding exon. In other words, the CDS of these genes is comprised in a single exon.

In some embodiments, the endogenous gene is selected from RAG1, RAG2, BTK, AAVS1, CD40L, CYBB, FOXP3, IL2RG, MAGT1, NCF1, and WAS.

In some embodiments, the endogenous gene is a RAG gene. In some embodiments, the endogenous gene is RAG1 or RAG2 gene. In some embodiments, the endogenous gene is RAG1 gene. In some embodiments, the endogenous gene is RAG2 gene.

In some embodiments, the endogenous gene is a human gene. In some embodiments, the endogenous gene is a human RAG gene, such as a human RAG1 gene or a human RAG2 gene.

The gene portion sequence defines a sequence which is part of the sequence of the endogenous gene that is to be replaced by the transgene, according to the invention. To aid the discussion, the structure of the CDSR construct will first be explained.

The CDSR construct comprises a transgene sequence for replacing the gene portion sequence, which is flanked by left (upstream) and right (downstream) homology arms, LHA, and RHA, respectively.

Accordingly, the 3′ end of the LHA is directly linked to the 5′ end of the transgene, and the 3′ end of the transgene is directly linked to the 5′ end of the RHA, as can be seen, e.g., in FIG. 1A. In a parallel description, the endogenous gene comprises the gene portion sequence (to be replaced by the transgene), flanked on the upstream side by a sequence that is essentially identical to the LHA, and on the downstream side by a sequence that is essentially identical to the RHA. In other words, the 5′ end of the gene portion sequence is linked to the 3′ end of the endogenous sequence that is essentially identical to the LHA, and the 3′ end of the gene portion sequence is linked to the 5′ end of the endogenous sequence that is essentially identical to the RHA.

Importantly, while the LHA and RHA are essentially identical to the corresponding endogenous sequence, in order to serve as templates for the HDR, the transgene sequence is not identical to the gene portion sequence it replaces.

In fact, the border between the transgene and the homology arms in the CDSR construct may be defined by the identity to the endogenous sequence, so that the flanking essentially identical sequences are the LHA and RHA, and the internal non-identical sequence is the transgene.

Accordingly, in some embodiments, the gene portion sequence 5′ and 3′ ends may be defined by comparing the endogenous gene sequence to the CDSR construct sequence. Based on the above explanation, the genomic sequence that lies between the upstream (5′) stretch of essentially identical sequence and downstream (3′) stretch of essentially identical sequence is defined as the gene portion sequence.

According to some embodiments, the gene portion sequence is comprised in one exon, and does not include intronic sequences or regulatory sequences. In some embodiments, the gene portion sequence does not comprise intronic sequences. In some embodiments, the gene portion sequence does not comprise regulatory sequences.

In some embodiments, the gene portion sequence comprises at least 10%, 20%, 30%, 40%, 50%, 60%, 70%, 80%, 90%, or 95% of the CDS of the endogenous gene. In some embodiments, the gene portion sequence comprises the complete CDS of the endogenous gene.

In some embodiments, the gene portion sequence comprises at least 10%, 20%, 30%, 40%, 50%, 60%, 70%, 80%, 90%, or 100% of the exon sequence.

In some embodiments, the gene portion sequence has a length of about 1-10000 bp. In some embodiments, the gene portion sequence has a length of about 1-5000 bp, 1-3500 bp, 1-2500 bp, 1-2000 bp, 1-1500 bp, 10-5000 bp, 10-3500 bp, 10-2000 bp, 100-5000 bp, 100-3500 bp, 100-2000 bp, 500-5000 bp, 500-3500 bp, or 500-2000 bp.

In some embodiments, the gene portion sequence comprises only coding sequence. In other words, in some embodiments, the gene portion sequence does not comprise non-coding sequences. In some embodiments, the gene portion sequence does not comprise 5′-untranslated region (UTR) sequences. In some embodiments, the gene portion sequence does not comprise 3′-UTR sequences. In some embodiments, the gene portion sequence comprises 5′-untranslated region (UTR) sequences. In some embodiments, the gene portion sequence comprises 3′-UTR sequences.

According to the invention, replacing the gene portion sequence with the transgene sequence is conducted by first generating a DSB at a desired location (the cleavage site), in the gene portions sequence or close to it. The DSB is generated by a genome editing system such as zinc finger nucleases (ZFNs), transcription-activator like effector nucleases (TALEN), meganucleases, and the clustered regularly interspaced short palindromic repeats (CRISPR/Cas) system. The DSB triggers cellular repair systems including non-homology end joining (NHEJ) and homology-directed repair (HDR). The HDR system repairs the DSB by using the CDSR constructs comprising the transgene flanked by homology arms as templates, thereby replacing the gene portion sequence with the transgene sequence.

The DSB (also referred to herein as a cleavage site) may be located at any suitable place with respect to the gene portion sequence, preferably close to or inside of the gene portion sequence.

When the DSB is outside of the gene portion sequence, it is located, by definition, in one of the homology arms. In some embodiments, there may be an advantage for the DSB to be close to the gene portion sequence, since when the DSB is too far from the gene portion sequence, long homology arms are needed to cover the distance between the DSB and the gene portion sequence. In such cases, the HDR may prematurely terminate. Accordingly, the DSB location is defined as being in a region spanning the gene portion sequence. Additionally, it is noted that at least with current CRISPR-Cas9-based systems, it is unlikely that the DSB may be at a distance of more than about 17 nucleotides from the gene portion sequence, since this will recreate the guide sequence site and may cause Cas9 to cut the repaired sequence.

The term “a region spanning the gene portion sequence” means either in the gene portion sequence, or in the vicinity of the gene portion sequence.

In some embodiments, the DSB is located in the LHA no more than about 5 kb upstream of the gene portion sequence. In some embodiments, the DSB is located in the LHA, no more than about 4000, 3000, 2000, 1000, 800, 500, 400, 300, 200, 100, 80, 50, 40, 30, 20, 15, 10, or 5 bp upstream of the gene portion sequence 5′ end.

In some embodiments, the DSB is located in the RHA, no more than about 5 kb downstream of the gene portion sequence. In some embodiments, the DSB is located in the RHA, no more than about 4000, 3000, 2000, 1000, 800, 500, 400, 300, 200, 100, 80, 50, 40, 30, 20, 15, 10, or 5 bp downstream of the gene portion sequence 3′ end.

Accordingly, in some embodiments, the region spanning the gene portion sequence is identical to the gene portion sequence. In some embodiments, the region spanning the gene portion sequence encompasses a sequence of about 5000, 4000, 3000, 2000, 1000, 800, 500, 400, 300, 200, 100, 80, 50, 40, 30, 20, 15, 10, or 5 bp both upstream and downstream of the gene portion sequence.

When the DSB is located inside the gene portion sequence, its position within the gene portion sequence determines its distance from the sequence that is essentially identical to the LHA and the RHA, which directly flank the gene portion sequence. There may be a risk that when it is too far, the transgene sequence that is not sufficiently identical to the gene portion sequence may not end up serving as template for the repair system, which may use other homologous sequences, such as the second allele.

Accordingly, in some embodiments, the DSB is located in the gene portion sequence.

In some embodiments, the DSB is located in the gene portion sequence, within no more than about 5 kb from the 5′ end of the gene portion sequence. In some embodiments, the DSB is located in the gene portion sequence, within no more than about 4000, 3000, 2000, 1000, 800, 500, 400, 300, 200, 100, 80, 50, 40, 30, 20, 15, 10, or 5 bp from the 5′ end of the gene portion sequence.

Accordingly, in some embodiments, the DSB is located in the gene portion sequence, within no more than about 5 kb from the 3′ end of the gene portion sequence. In some embodiments, the DSB is located in the gene portion sequence, within no more than about 4000, 3000, 2000, 1000, 800, 500, 400, 300, 200, 100, 80, 50, 40, 30, 20, 15, 10, or 5 bp from the 3′ end of the gene portion sequence.

The phrase “at least one genome editing reagent” refers to a reagent or reagents used with genome editing systems. Such reagents may include a nuclease, a guide RNA, and/or any other reagents needed for a particular system. In some embodiments, the at least one genome editing reagent is suitable for use with a genome editing system selected from zinc finger nucleases (ZFNs), transcription-activator like effector nucleases (TALEN), meganucleases, and an clustered regularly interspaced short palindromic repeats (CRISPR/Cas) system. In some embodiments, the genome editing system is a CRISPR/Cas system. In some embodiments, the genome editing system is a CRISPR/Cas9 system.

The CRISPR/Cas9 system includes a guide RNA, such as a single guide RNA (sgRNA) for directing the CRISPR/Cas9 system to the desired position of the DSB. Accordingly, in some embodiments, the at least one genome editing reagent comprises a guide RNA. In some embodiments, the at least one genome editing reagent comprises an sgRNA. In some embodiments, the at least one genome editing reagent comprises a nuclease. In some embodiments, the at least one genome editing reagent comprises a nuclease and a guide RNA. In some embodiments, the at least one genome editing reagent comprises a CRISPR-Cas9 RNP complex comprising Cas9 and an sgRNA.

In some embodiments, the guide RNA has a length of about 15-30 nucleotides. In some embodiments, the guide RNA has a length of about 17-24 nucleotides. In some embodiments, the guide RNA has a length of about 20 nucleotides.

In some embodiments, the guide RNA, or the sgRNA, is essentially identical to a sequence of the endogenous gene which includes the cleavage site, or the DSB.

The sgRNAs exemplified in the present application is an sgRNA used for gene editing of the RAG2 and the RAG1 genes according to the invention, having sequences as defined in SEQ ID NO: 1: UGAGAAGCCUGGCUGAAUUA (RAG2) and SEQ ID NO: 2: UUGACUCAGGGUUCCACCCA (RAG1), respectively.

The CDSR construct is generally described above and will be described below in more detail. The CDSR construct may also be referred to as a KI construct, since it knocks-in the transgene sequence thereby replacing the gene portion sequence.

The length of the homology arms may vary based on several factors, including their distance from the DSB. The inventors have found that when a CDS replacement construct was designed by distancing the RHA from the DSB, HDR frequencies were reduced compared to the insertion construct with equivalent homology arm lengths (see FIGS. 1B and 1C). Recently, Cromer et al. (Cromer et al., 2021, Gene replacement of α-globin with β-globin restores hemoglobin balance in β-thalassemia-derived hematopoietic stem and progenitor cells. Nat Med. 27(4):677-687) showed that extending the length of the homology arms can lead to higher rates of HDR. Thus, to increase the efficiencies with the CDS replacement construct, the RHA (which is further away from the DSB) was extended, which induced higher HDR frequencies. It is hypothesized that increasing the length of the distal homology arm may enhance the probability of recognition and subsequent incorporation of the exogenous construct template by the cellular HDR mechanism.

In some embodiments, the homology arm that is more distant from the DSB is longer.

In some embodiments, the distance between the LHA and the DSB is longer than the distance between the RHA and the DSB and the LHA is longer than the RHA.

In some embodiments, the distance between the RHA and the DSB is longer than the distance between the LHA and the DSB and the RHA is longer than the LHA.

In some embodiments, the distance between the LHA and the DSB is less than about 5000, 4000, 3000, 2000, 1000, 800, 500, 400, 300, 200, 100, 80, 50, 40, 30, 20, 10, or 5 bp.

In some embodiments, the distance between the RHA and the DSB is less than about 5000, 4000, 3000, 2000, 1000, 800, 500, 400, 300, 200, 100, 80, 50, 40, 30, 20, 10, or 5 bp.

In some embodiments, the distance between the LHA or the RHA and the DSB is less than about 5000, 4000, 3000, 2000, 1000, 800, 500, 400, 300, 200, 100, or 80 bp. In some embodiments, the distance between the LHA or the RHA and the DSB is less than about 50, 40, 30, or 20 bp. In some embodiments, the distance between the LHA or the RHA and the DSB is less than about 10 or 5 bp.

In some embodiments, the length of the LHA and/or the RHA is about 20-5000 bp.

In some embodiments, the length of the LHA is about 50-4000, 100-3000, 200-2500, 200-2000, 200-1500, 200-1000, 200-800, 400-3000, 400-2500, 400-2000, 400-1500, 400-1000, or 400-800 bp.

In some embodiments, the length of the RHA is about 20-5000, 50-4000, 100-3000, 200-2500, 200-2000, 200-1500, 200-1000, 200-800, 400-3000, 400-2500, 400-2000, 400-1500, 400-1000, or 400-800 bp.

Some examples for LHAs demonstrated herein include SEQ ID Nos: 10 and 15 (and see complete list below).

Some examples for RHAs demonstrated herein include SEQ ID Nos: 12-14, and 17-19 (and see complete list below).

The transgene replacing the gene portion sequence may include various elements.

First, the transgene includes an edited sequence, which is a corrected, or edited, version of the gene portion sequence. By definition, the edited sequence is different from the gene portion sequence. The edited sequence may generally be based on a wild-type genomic sequence corresponding to the gene portion sequence to be edited, but may include further modifications. It is also conceivable that the edited sequence may be based on a different sequence which is desired to replace the gene portion sequence.

When the edited sequence is a protein-coding sequence based on the wild-type genomic sequence, the edited sequence may include variations, such as wobble of the amino acid codons. As a result, the edited sequence may encode the same amino acid sequence as the wild-type genomic sequence, but by using different amino acid codons. Using different amino acid codons compared to the wild-type sequence also causes the edited sequence to be more different from the gene portion sequence, thereby decreasing the likelihood of premature termination of the HDR. It is also conceivable that the edited sequence may encode an amino acid sequence that is not identical to the amino acid sequence encoded by the wild-type genomic sequence.

The terms “wild-type genomic sequence” and “wild-type amino acid sequence” relate to wild-type (normal, healthy, unmutated, or functional) genomic sequence corresponding to the gene portion sequence, and the corresponding amino acid sequence encoded by it.

In some embodiments, the edited sequence comprises a sequence having at least about 60%, 70%, 80%, 90%, 95%, 96%, 97%, 98%, or 99% identity to a wild-type genomic sequence corresponding to the gene portion sequence.

In some embodiments, the edited sequence comprises a sequence having at most about 70%, 80%, 90%, 95%, 96%, 97%, 98%, or 99% identity to a wild-type genomic sequence corresponding to the gene portion sequence.

In some embodiments, the edited sequence comprises a sequence having at least about 60%, 70%, 80%, 90%, 95%, 96%, 97%, 98%, or 99% identity to the gene portion sequence.

In some embodiments, the edited sequence comprises a sequence having at most about 70%, 80%, 90%, 95%, 96%, 97%, 98%, or 99% identity to the gene portion sequence.

In some embodiments, the edited sequence comprises a CDS.

In some embodiments, the edited sequence comprises only coding sequence.

In some embodiments, the edited sequence does not comprise endogenous 5′ or 3′ UTR sequences. In some embodiments, the edited sequence comprises endogenous 5′ UTR sequences. In some embodiments, the edited sequence comprises endogenous 3′ UTR sequences.

In some embodiments, the edited sequence comprises a CDS encoding the same amino acid sequence as a wild-type sequence corresponding to the gene portion sequence.

In some embodiments, the edited sequence comprises a CDS encoding an amino acid sequence having at least 80%, 90%, 95%, 96%, 97%, 98%, or 99% identity to a wild-type amino acid sequence encoded by the wild-type genomic sequence.

In some embodiments, the CDS of the edited sequence is not identical to the CDS of the wild-type genomic sequence, but it encodes an amino acid sequence that is identical to the wild-type amino acid sequence.

In some embodiments, the CDS of the edited sequence includes at least one amino acids codon that is different from an amino acid codon used in the wild-type genomic sequence for the same amino acid at a corresponding position in the wild-type amino acid sequence.

An exemplary edited sequence according to the invention is dcoRAG2, set forth in SEQ ID NO: 20.

The transgene may include further elements, such as upstream or downstream elements for enhancing transcription or reporting elements for follow-up on the success of HDR.

Polyadenylation at the end of a RNA transcript affects mRNA stability, nuclear export, and translation, thus, playing a crucial role in gene expression. In vitro studies using rAAV6 vectors have highlighted the effects of various synthetic polyA signals, such as BGH polyA signal sequences and WPRE in boosting transgene expression. Such elements may be helpful for nuclear export of intronless RNA, and the addition of such elements has been reported to enhance transgene expression in vitro.

In some embodiments, the transgene further comprises at least one element upstream of the CDS, which is not present in the endogenous gene.

In some embodiments, the transgene further comprises at least one element downstream of the CDS, which is not present in the endogenous gene.

In some embodiments, the transgene further comprises at least one 3′ element selected from: a partial or complete endogenous 3-UTR of the endogenous gene, a woodchuck hepatitis virus posttranscriptional regulatory element (WPRE), and/or a poly A sequence such as a bovine growth hormone poly A (BGHpA) sequence or an SV40 polyA sequence.

The present invention also provides means for following up on the success and accuracy of the HDR process and for enriching for the targeted cell population by using at least one KI reporter gene. The KI reporter gene may be any suitable reporter gene which produces a reporter gene product that may be detected by any suitable method. However, KI reporter genes suitable for use in cells intended for gene therapy (i.e., not experimental systems) should be compatible with administration to a human subject.

Examples for reporter genes suitable for editing cells intended to be administered to a human subject include truncated cell-surface markers, which are easy to detect, e.g., with specific antibodies, by using flow cytometry or similar methods, may be used to enrich cells from a heterogeneous population, and enrichment may be done in a single step. Specific examples for such truncated cell-surface markers for use with the invention include truncated nerve growth factor receptor (tNGFR); CD19t, a truncated version of CD19; CD34t, a truncated version of CD34; EGFRt, a truncated version of EGFR; HER2t, a truncated version of HER2; and CD49t, a truncated version of CD49a. Other truncated cell-surface markers may also be used.

It is noted that for experimental systems, additional reporter genes (not necessarily compatible with human administration) may be used, such as those listed below, with reference to KO reporter genes.

The KI reporter gene may be provided with an exogenous promoter such as PGK promoter, ubiquitin C (UBC) promoter, and eukaryotic translation elongation factor 1 (EF1) promoter, in which case its expression does not depend on the endogenous gene expression.

However, the KI reporter gene may be designed so as to be expressed as part of the edited gene, depending on and reflecting the activity of the endogenous promoter. Furthermore, the present invention provides a system in which the KI reporter gene is inserted in frame into, or at the end of, the CDS of the edited gene, such that the reporter gene product is translated as part of the edited gene product. For example, the KI reporter gene may be inserted instead of the stop codon of the edited gene CDS, or by replacing a short sequence at the 3′ end of the edited gene CDS, just before the stop codon.

The reporter gene may also be designed to be cleaved, or self-cleaved, to release the reporter gene product following translation. It is appreciated that when the KI reporter gene is translated as part of the edited gene product and then cleaved to release the reporter gene product, there is a 1:1 ratio between the amount of KI reporter gene product and the amount of the edited gene product and so the level of the KI reporter gene product is indicative of the endogenous promoter activity and the level of the edited gene product. In this way, the level and timing of expression of the KI reporter gene product directly reflects the expression of the edited gene product. Further, this method eliminates the need to incorporate a potentially problematic external constitutive promoter to drive expression of the reporter.

In some embodiments, the transgene further comprises a sequence comprising a KI reporter gene sequence encoding a KI reporter gene product. In some embodiments, the KI reporter gene is located downstream of the edited sequence. In some embodiments, the KI reporter gene is located upstream of the edited sequence.

In some embodiments, the KI reporter gene product is a truncated cell-surface marker. In some embodiments, the KI reporter gene product is selected from truncated nerve growth factor receptor (tNGFR), CD19t, CD34t, EGFRt, HER2t, and CD49t.

In some embodiments, the transgene is designed such that the edited sequence and the KI reporter gene sequence form a single CDS which encodes the edited gene product including the reporter gene product. For example, the transgene is designed such that the KI reporter gene CDS is incorporated in frame at the 3′ end of the CDS of the edited sequence. Accordingly, in some embodiments, the edited gene product comprises the reporter gene product.

In some embodiments, the KI reporter gene product comprises a cleavable site designed to release the KI reporter gene product from the edited gene product. In some embodiments, the edited gene product initially comprises the KI reporter gene product, which is subsequently cleaved off from the edited gene product.

In some embodiments, the KI reporter gene product is self-cleavable. In some embodiments, the KI reporter gene product comprises a self-cleaving peptide. Examples for self-cleaving peptides include 2A peptides such as P2A, E2A, F2A, and T2A. In some embodiments, the self-cleaving peptide is a T2A peptide.

In some embodiments, the self-cleaving peptide is followed by the reporter gene product. In other words, in some embodiments, the self-cleaving peptide sequence is at the amino side of the gene product relative to the reporter gene product, such that after translation of the edited product comprising the KI reporter gene product, cleavage at the self-cleaving peptide sequence releases the reporter gene product from the edited gene product.

In some embodiments, the KI reporter gene product comprises a self-cleaving peptide linked to a truncated cell-surface marker. In some embodiments, the KI reporter gene encodes a self-cleaving peptide followed by a truncated cell-surface marker

A specific example for such a system which is used herein is the T2A-tNGFR system including a T2A self-cleaving peptide followed by a truncated nerve growth factor receptor (tNGFR), such that cleavage at the T2A peptide sequence releases the tNGFR from the edited gene product.

Accordingly, in some embodiments, the transgene further comprises a T2A-tNGFR system encoded with the edited gene product as part of a single gene product.

In some embodiments, the transgene has a length of about 10-4000, 10-3500, 10-3000, 10-2500, 10-2000, 10-2500, 10-2000, 10-1500 or 10-1000 bp. In some embodiments, the transgene has a length of about 50-4000, 50-3500, 50-3000, 50-2500, 50-2000, 50-2500, 50-2000, 50-1500 or 50-1000 bp. In some embodiments, the transgene has a length of about 100-4000, 100-3500, 100-3000, 100-2500, 100-2000, 100-1500, or 100-1000 bp.

Exemplary CDSR constructs according to the invention include SEQ ID Nos: 29-31.

The CDSR construct may be incorporated into a suitable delivery vector for delivery of the construct into a cell, such as a cell of a subject. Such delivery vectors may include any suitable vector for delivery of DNA to mammalian cells, such as viral, retroviral, lentiviral, adenoviral, or adeno-associated viral (AAV) vectors. Examples for suitable vectors include adenovirus 5/35 serotype (AdV), recombinant adeno-associated virus serotype 6 (rAAV6), and integration-deficient lentivirus (IDLV).

Alternatively, the CDSR constructs may be delivered as naked single-stranded oligodeoxynucleotides (ssODNs) or single-stranded DNA (ssDNA).

Accordingly, in some embodiments, the replacement nucleic acid molecule further comprises a delivery vector, for delivery of the CDSR construct into a cell.

In some embodiments, the delivery vector is a rAAV6.

In some embodiments, the replacement nucleic acid molecule does not comprise a delivery vector.

In some situations, both alleles of the endogenous gene must be replaced. An example may be a disease caused by a dominant allele. By only carrying out the KI strategy outlined above, it is difficult to confirm that both endogenous alleles of the endogenous gene have been replaced. Other reasons may also exist for preferring to replace both endogenous alleles, one example being generating an experimental system for simulating the function of a mutated allele, or for simulating gene editing when starting from unmutated cells, such as healthy donor (HD)-derived cells (as also exemplified herein).

For this purpose, the present invention further provides a KO construct to be used together with the KI construct. The KO construct may be any suitable construct designed to knock-out or replace the endogenous gene or a part thereof. The KO construct may include a reporter gene (KO reporter gene) which is designed to integrate into the endogenous gene and is different from the KI reporter gene, so that it is possible to enrich for cells which incorporated both KO and KI reporters (one in each allele).

The KO construct may be designed for being incorporated by any suitable gene knock-out method, such as gene editing.

In some embodiments, the KO construct is designed to be incorporated by the same method used for incorporating the KI construct. In some embodiments, the KO construct is designed to be incorporated by gene editing. In some embodiments, the KO construct is designed to be incorporated by HDR triggered by a DSB generated by a CRISPR/Cas9 system.

Accordingly, in some embodiments, the system further comprises a KO nucleic acid molecule comprising a KO construct which comprises a KO reporter gene sequence encoding a KO reporter gene product, wherein the KO construct is designed for incorporating the KO reporter gene sequence into an integration region in the endogenous gene sequence, thereby preventing expression of an endogenous gene product.

The term “integration region” means a position in the endogenous gene sequence into which the KO reporter gene is designed to integrate (by insertion), or a sequence in the endogenous gene sequence which is designed to be replaced by the KO reporter gene.

The term “endogenous gene product” relates to a gene product of the endogenous gene. The endogenous gene product may be a product of any one of the two alleles.

An example of such a KO construct which has a structure similar to the structure of the CDSR construct, and that is suitable for use in the methods of the invention, including sequence replacement by HDR, is provided below.

In some embodiments, the KO construct comprises:
a. a KO left homology arm (KO-LHA) comprising a sequence essentially identical to a sequence directly upstream of the integration region;
b. a KO right homology arm (KO-RHA) comprising a sequence essentially identical to a sequence directly downstream of the integration region; and
c. a KO reporter gene positioned between the KO-LHA and the KO-RHA,
wherein following incorporating the KO reporter gene, the endogenous gene expresses the KO reporter gene product instead of the endogenous gene product.

The KO reporter gene may be any suitable reporter gene, including reporter genes suitable for gene therapy, as noted above for the KI reporter gene, including truncated cell-surface markers such as truncated nerve growth factor receptor (tNGFR); CD19t, a truncated version of CD19; CD34t, a truncated version of CD34; EGFRt, a truncated version of EGFR; HER2t, a truncated version of HER2; and CD49t, a truncated version of CD49a. Other truncated cell-surface markers may also be used.

In some embodiments, the KO reporter gene is different from the KI reporter gene.

Additionally, the KO reporter gene may be any reporter gene for in vitro use. Nonlimiting examples include green fluorescent protein (GFP), blue fluorescent protein (BFP), yellow fluorescent protein (YFP), citrine, mCherry, enhanced green fluorescent protein (EGFP), etc.

In some embodiments, the KO reporter gene further comprises an exogenous promoter. Examples for suitable promoters include gene-therapy-compatible promoters such as PGK, UB1, and EF1, as well as promoters which are not necessarily suitable for therapy such as spleen focus-forming virus (SFFV).

In some embodiments, the KO reporter gene is designed to be incorporated by replacing the endogenous gene or a partial sequence thereof. In some embodiments, the KO reporter gene is designed to be incorporated by insertion into the endogenous gene sequence.

In some embodiments, the reporter gene further includes upstream or downstream elements similar to elements described for the transgene of the CDSR construct. Examples for such elements include BGHpA and/or WPRE.

In some embodiments, the KO-LHA is identical to the LHA of the CDSR construct.

In some embodiments, the KO-RHA is identical to the RHA of the CDSR construct.

In some embodiments, the KO-LHA is different from the LHA of the CDSR construct.

In some embodiments, the KO-RHA is different from the RHA of the CDSR construct.

Exemplary KO constructs include SEQ ID Nos: 23-27 (RAG2) and 33-35 (RAG1).

Similar to the CDSR construct, the KO construct may also be incorporated into a suitable delivery vector for delivery of the construct into a cell, such as a cell of a subject. Such delivery vectors may include any suitable vector for delivery of DNA to mammalian cells, such as viral, retroviral, lentiviral, adenoviral, or AAV vectors. Examples for suitable vectors include adenovirus 5/35 serotype (AdV), rAAV6, and IDLV.

Alternatively, the KO constructs may be delivered as naked ssODNs or ssDNA.

Accordingly, in some embodiments, the KO nucleic acid molecule further comprises a delivery vector into which the KO construct is incorporated. In some embodiments, the KO nucleic acid molecule does not comprise a delivery vector.

In some embodiments, the delivery vector is rAAV6.

The present invention further provides a method for using the above system.

In some embodiments, there is provided an ex vivo or in vitro method for editing of an endogenous gene by replacing a gene portion sequence thereof comprised in a single exon with a transgene sequence, the method comprising:
a. providing stem cells or progenitor cells suitable for gene targeting;
b. contacting the cells with at least one genome editing reagent thereby generating a DSB in a region spanning the gene portion sequence;
c. contacting the cells with a replacement nucleic acid molecule comprising a coding sequence replacement (CDSR) construct serving as a template for homology-directed repair (HDR) triggered by the DSB, the CDSR construct comprising:
   i. a left homology arm (LHA) comprising a sequence essentially identical to a sequence directly upstream of the gene portion sequence,
   ii. a right homology arm (RHA) comprising a sequence essentially identical to a sequence directly downstream of the gene portion sequence, and
   iii. the transgene sequence comprising an edited sequence and positioned between the LHA and the RHA; and
d. isolating edited cells comprising an edited gene encoding an edited gene product.

It is appreciated that since the methods of the invention use the systems of the invention described above, definitions of terms relating to the systems also apply to the same terms when used in the methods, and embodiments detailed above with reference to system elements and features, also apply to the corresponding elements and features when mentioned in the methods. Some embodiments may be repeated below, for clarity and flow.

Unless noted otherwise, the term “cells” when mentioned without further specification of the type of cells, is meant to relate to the cell precursors of the method, i.e., the stem cells or progenitor cells.

The process of gene editing by using at least one genome editing reagent is explained in detail above. Contacting the cells with at least one genome editing reagent may be done by any suitable method of introducing protein or protein/nucleic acid complexes into cells, such as by electroporation, or nanoparticles-mediated transfection, e.g. with lipid nanoparticles.

In some embodiments, contacting the cells with at least one genome editing reagent in step (b) is conducted by electroporating the cells with the at least one genome editing reagent. In some embodiments, the contacting in step (b) is conducted by electroporating the cells with the at least one genome editing reagent protein complexes or protein/nucleic complexes.

In some embodiments, the genome editing system is a CRISPR/Cas9 system including a guide RNA (or an sgRNA), and step (b) comprises forming a Cas9/sgRNA RNP complex and delivering the RNP complex into the cells, for example, by electroporation. In some embodiments, the contacting in step (b) is conducted by electroporating the cells with a CRISPR/Cas9 RNP complex comprising Cas9 complexed with a single-guide RNA (sgRNA).

In some embodiments, contacting the cells in step (c) may be conducted by any suitable method for delivery of nucleic acids into cells. Nonlimiting examples for such methods include electroporation, transfection, and transduction.

In some embodiments, when the replacement nucleic acid molecule comprises viral or retroviral vector sequences, contacting the cells in step (c) is conducted by transduction the cells with the replacement nucleic acid molecule.

When using viral or retroviral sequences it is generally advantageous to lower the multiplicity of infection (MOI) in order to prevent or lower potential toxicity.

In some embodiments, the replacement nucleic acid molecule comprises a viral or retroviral vector sequence, and the transduction is conducted at an MOI of less than 30000, 25000, 20000, 15000, 10000, 5000, 3000, 2000, 1000, 500 or 200 viral genomes/cell. In some embodiments, the replacement nucleic acid molecule comprises a viral or retroviral vector sequence, and the transduction is conducted at an MOI of less than 20000, or 15000 viral genomes/cell.

In some embodiments, the replacement nucleic acid molecule comprises a viral or retroviral vector sequence, and the transduction is conducted at an MOI of about 200-100000 viral genomes/cell. In some embodiments, the transduction is conducted at an MOI of about 200-50000, 200-30000, 200-20000, 200-15000, 200-10000, 500-15000, 1000-15000, 5000-20000, 5000-30000, 5000-25000, 5000-20000, 5000-15000, 10000-50000, 10000-30000, 10000-25000, 10000-20000, or 10000-15000 viral genomes/cell.

As explained above with reference to the system, the transgene may further comprise additional elements, such as 5′ or 3′ regulatory elements, or a reporter gene sequence.

Certain specific embodiments of the system may relate to some variations of the methods and will be described here in more detail.

In some embodiments, the transgene further comprises a sequence comprising a KI reporter gene sequence encoding a KI reporter gene product.

The KI reporter gene product may be detected by any suitable method, in order for following up on the HDR and for enriching for positive cells. In some embodiments, the KI reporter gene product is a truncated cell-surface marker. In some embodiments, the KI reporter gene product is selected from truncated nerve growth factor receptor (tNGFR), CD19t, CD34t, EGFRt, HER2t, and CD49t. Such cell-surface markers may easily be detected or used for enrichment of cells by using specific antibodies to these markers in methods such as fluorescence activated cell sorting (FACS) or similar methods known in the art for enriching cells by using antibodies.

Accordingly, in some embodiments, isolating edited cells in step (d) comprises enriching for cells expressing the KI reporter gene product. In view of the above, the enrichment may be conducted by any suitable enrichment method based on using antibodies against the truncated cell-surface markers, including, e.g., FACS, magnetic beads, and other commonly used methods.

In some embodiments, the transgene is designed such that the edited sequence and the KI reporter gene sequence form a single CDS which encodes the edited gene product including the KI reporter gene product. As explained above, this may be designed, for example, by incorporating the KI reporter gene in frame at the end of the edited sequence CDS, before the stop codon. In some embodiments, the KI reporter gene product comprises a cleavable site designed to release the KI reporter gene product from the edited gene product.

In some embodiments, the KI reporter gene product is self-cleavable. In some embodiments, the KI reporter gene product comprises a self-cleaving peptide. Examples for self-cleaving peptides include 2A peptides such as P2A, E2A, F2A, and T2A. In some embodiments, the self-cleaving peptide is a T2A peptide.

In some embodiments, the KI reporter gene product comprises a self-cleaving peptide followed by a truncated cell-surface marker. In some embodiments, the KI reporter gene encodes a self-cleaving peptide followed by a truncated cell-surface marker.

In some embodiments, the KI reporter gene product comprises a T2A-tNGFR system including a T2A self-cleaving peptide followed by a truncated nerve growth factor receptor (tNGFR).

It is appreciated, that in some cases the expression of the KI reporter gene may not start immediately following the gene editing. Such cases may be, for example, when expression of the KI reporter gene is driven by a promoter of the endogenous gene, such as in the case when the KI reporter gene is expressed as part of the edited gene product. The endogenous gene promoter may be only activated in a specific cell type or differentiation stage, and not be active in the precursor stem cells or progenitor following editing. In this case, the cells must be grown and differentiated in vitro so as to induce activity of the endogenous gene promoter, before the reporter gene can be detected and used for enrichment.

This is also the case for the RAG1 and RAG2 genes, which are expressed at a specific stage in B and T cell differentiation, when V(D)J recombination occurs, and therefore their promoters are not active in stem cells or progenitor cells. According to the methods of the invention, the cells are grown in an in vitro differentiation system at least until the RAG1 or RAG2 promoter is activated, which then facilitates enriching for edited cells. In some embodiments, the cells are grown in an in vitro differentiation system also after the endogenous promoter is activated.

Accordingly, in some embodiments, isolating edited cells in step (d) further comprises growing the cells under differentiation conditions at least until the endogenous promoter is activated and the KI reporter gene is expressed, and enriching for cells expressing the KI reporter gene product. The differentiation conditions may depend on the type of cells used and are any differentiation conditions suitable for inducing activity of the endogenous gene promoter.

In some embodiments, when the KI reporter gene is expressed as part of the edited gene product and the endogenous gene is not expressed in the stem cells or progenitor cells, the isolating in step (d) further comprises growing the cells under differentiation conditions suitable for inducing expression of the endogenous gene, and enriching for cells expressing the KI reporter gene product.

It is noted that the phrases “inducing expression of the endogenous gene” and “inducing activity of the endogenous gene promoter” may be used interchangeably. Further, conditions suitable for inducing expression of the endogenous gene are intended to be the same conditions suitable for inducing expression of the edited gene, since the promoter is the same promoter.

In some embodiments, growing the cells under differentiating conditions is conducted at least until the KI reporter gene is expressed. In some embodiments, growing the cells under differentiating conditions is conducted at least until certain differentiation markers are expressed. In some embodiments, growing the cells under differentiating conditions is conducted until the KI reporter gene is expressed. In some embodiments, growing the cells under differentiating conditions is conducted until certain differentiation markers are expressed. The differentiation markers may be defined based on the desired cell type.

Differentiation markers suitable for T cell differentiation include CD34, CD7, CD5, CD1a, CD3, CD4, CD8, and TCRγδ.

In some embodiments, the differentiation conditions are T cell differentiation conditions. It is appreciated that differentiation conditions are known for many cell types, and particularly for T cells, and systems and kits for differentiation of T cells and other cell types are available (e.g., for T cells: Boyd et al., 2021, Cells 10(10):2631; for B cells: Richardson et al., 2021, STAR Protoc. 2(2):100420 (PMID: 33899010); for NK cells: Luevano et al., 2012, Cell Mol Immunol. 9(4):310-20 (PMID: 22705914); and for other hematopoietic cells: Bozhilov et al., 2023 Cells 12(6):896 (PMID: 36980237)).

The method of the invention may further involve a combination of the CDSR (or KI) construct and a KO construct, as noted above for the system. This strategy may be used for knocking out both alleles in a case of a dominant disease, and it may also be used in order to set up an experimental system using healthy donor (HD) cells. The important feature of the combination of KI and KO constructs is in that two different reporter genes are used for the KI and the KO reporter genes, and the edited cells are isolated by enriching for expression of both the KI and the KO reporter products, thereby allowing to make sure that both alleles have been edited.

In some embodiments, the method further comprises knocking-out the endogenous gene.

In some embodiments, the method further comprises contacting the cells with a KO nucleic acid molecule comprising a KO construct which comprises a KO reporter gene encoding a KO reporter gene product, wherein the KO construct is designed for incorporating the KO reporter gene into an integration region in the endogenous gene sequence, thereby preventing expression of an endogenous gene product, and the KO reporter gene is different from the KI reporter gene.

In some embodiments, contacting the cells with a KO nucleic acid molecule is conducted at step (c). In some embodiments, contacting the cells with a KO nucleic acid molecule is conducted at step (c), and the cells are contacted with both the KO nucleic acid molecule and the replacement nucleic acid molecule at the same time. In some embodiments, contacting the cells with a KO nucleic acid molecule is conducted separately from the contacting with the replacement construct. In some embodiments, contacting the cells with a KO nucleic acid molecule is conducted before step (c).

In some embodiments, isolating the cells in step (d) comprises enriching for cells expressing both the KI reporter gene product and the KO reporting gene product.

In some embodiments, such as when the KI reporter gene is designed to be driven by the endogenous promoter and the KO reporter gene is designed to be driven by an external constitutive promoter, isolating edited cells in step (d) includes first enriching for cells positive for the KO reporter gene product and then growing the cells under differentiating conditions and enriching for cells positive for the KI reporter gene product.

In some embodiments, such as when both the KI and the KO reporter genes are driven by the endogenous promoter, isolating edited cells in step (d) includes growing the cells under differentiating conditions and enriching for cells positive for both the KI and the KO reporter gene products.

The cells used as precursors for the methods of the invention are generally multipotent cells such as stem cells or progenitor cells appropriate for development into the type of cells which need to be used for the therapy.

For endogenous genes expressed in the hematopoietic or immune system the appropriate stem or progenitor cells are hematopoietic stem and progenitor cells (HSPCs), such as CD34⁺ HSPCs.

In some embodiments, the stem cells or progenitor cells are hematopoietic or immune stem or progenitor cells.

In some embodiments, the stem cells or progenitor cells are CD34⁺ hematopoietic stem and progenitor cells (HSPCs).

In some embodiments, the stem cells or progenitor cells are multipotent stem cells.

In some embodiments, the stem cells or progenitor cells are pluripotential stem cells.

In some embodiments, the stem cells or progenitor cells are derived from a subject suffering from a disease or disorder caused by a mutation in the endogenous gene.

In some embodiments, the stem cells or progenitor cells are derived from a subject suffering from a disease or disorder caused by a mutation in the RAG1 or the RAG2 gene.

In some embodiments, the disease or disorder is selected from SCID, leaky SCID, chronic granulomatous disease (CGD), delayed onset combined immunodeficiency with granulomas and/or autoimmunity (CID-G/AI), Pelvic inflammatory disease (PID), enteropathy, X-linked (IPEX) syndrome, X-Linked Agammaglobulinemia (XLA), immune dysregulation, polyendocrinopathy, X-linked immunodeficiency with hyper-IgM (XHIM), X-linked MAGT1 deficiency with increased susceptibility to Epstein-Barr virus (EBV) infection and N-linked glycosylation (XMEN) syndrome, and Omenn syndrome. In some embodiments, the disease or disorder is SCID.

In some embodiments, the stem cells or progenitor cells are derived from a healthy donor.

In some embodiments, the method further comprises, before or after step (d), a step of differentiating the cells to a desired cell type. The desired cell type may be any cell type suitable for administration to a subject for the purpose of gene therapy.

In some embodiments, the method further comprises, before or after step (d) enriching for cells expressing markers indicative of cellular differentiation. Such markers may be typical to the desired cell type. In some embodiments, the markers indicative of cellular differentiation are selected from CD34, CD7, CD5, CD1a, CD3, CD4, CD8, and TCRγδ, indicative of T cell differentiation.

In some embodiments, the method further comprises adding non-homologous end joining (NHEJ) pathway inhibitors. In some embodiments, the NHEJ pathway inhibitors are added at step (b) of the method, when adding the at least one genome editing reagent to the cells. In some embodiments, the NHEJ inhibitors are SCR7 (DNA ligase IV inhibitor); i53 (an engineered ubiquitin variant, inhibitor of 53BP1); DNA-dependent protein kinase (DNA-PK) inhibitors such as AZD7648 and M3814; DNA polymerase theta inhibitors such as novobiocin (NVB); and/or p53 inhibitors such as GSE56.

In some embodiments, the present application provides an edited cell comprising an edited gene, obtained by the methods disclosed herein. The edited cell may be of any cell type, as disclosed above with reference to the methods.

In some embodiments, the present application provides a pharmaceutical composition comprising the edited gene disclosed herein, and a pharmaceutically acceptable carrier.

In some embodiments, the edited cell of the invention is a stem cell or a progenitor cell edited by the methods of the invention. In some embodiments, the edited cell of the invention is an edited cell that has gone through further differentiation following the isolation in step (d) of the method.

In some embodiments, there is provided a use of the system for editing of an endogenous gene disclosed herein, or of the edited cell comprising an edited gene disclosed herein, for treating a disease or disorder caused by a mutation in the endogenous gene in a subject in need thereof.

In some embodiments, there is provided a use of the system for editing of an endogenous gene disclosed herein, or of the edited cell comprising an edited gene disclosed herein, in the preparation of a medicament for treating a disease or disorder caused by a mutation in the endogenous gene in a subject in need thereof.

In some embodiments, there is provided the system for editing of an endogenous gene disclosed herein, or the edited cell comprising an edited gene disclosed herein, for use in treating a disease or disorder caused by a mutation in the endogenous gene in a subject in need thereof.

In some embodiments, there is provided a method of treating a disease or a disorder caused by a mutation in an endogenous gene in a subject in need thereof, comprising administering to the subject the pharmaceutical composition disclosed herein, which comprises the edited cells obtained by the methods disclosed herein, comprising the edited gene.

In some embodiments, there is provided a method of treating a disease or a disorder caused by a mutation in an endogenous gene in a subject in need thereof, by replacing a gene portion sequence of the endogenous gene comprised in a single exon with a transgene sequence, the method comprising:
a. providing stem cells or progenitor cells suitable for gene targeting;
b. contacting the cells with at least one genome editing reagent thereby generating a DSB in a region spanning the gene portion sequence;
c. contacting the cells with a replacement nucleic acid molecule comprising a coding sequence replacement (CDSR) construct serving as a template for homology-directed repair (HDR) triggered by the DSB, the CDSR construct comprising:
   i. a left homology arm (LHA) comprising a sequence essentially identical to a sequence directly upstream of the gene portion sequence,
   ii. a right homology arm (RHA) comprising a sequence essentially identical to a sequence directly downstream of the gene portion sequence, and
   iii. the transgene sequence comprising an edited sequence and positioned between the LHA and the RHA;
d. isolating edited cells comprising an edited gene encoding an edited gene product; and
e. administering to the subject the edited cells or a pharmaceutical composition comprising them.

In some embodiments, the stem cells or progenitor cells are autologous cells of the subject.

In some embodiments, the pharmaceutically acceptable carrier is a buffer, diluent, adjuvant, excipient, or vehicle suitable for administration with the cells of the invention. In some embodiments, the pharmaceutically acceptable carrier may be suitable for intravenous infusion. In some embodiments, the pharmaceutically acceptable carrier may be suitable as a cryoprotectant. In some exemplary embodiments, the carrier may be DMSO (for example, at about 10%). In some embodiments, the pharmaceutically acceptable carrier may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The term “treating”, as used herein, refers to means of obtaining a desired physiological effect, in this case, partially or completely curing the disease or disorder and/or symptoms thereof. The term may relate to ameliorating or inhibiting the disease or its symptoms, i.e. arresting its development or curing it completely.

The administration of the cells and/or compositions comprising them may be conducted by any suitable method for administration of cells, such as, but not limited to intravenous injection or infusion.

In some embodiments, there is provided a kit for editing of an endogenous gene by replacing a gene portion sequence thereof comprised in a single exon with a transgene sequence, the kit comprising:
a first receptacle comprising at least one genome editing reagent designed for generating a double strand break (DSB) in a region spanning the gene portion sequence; and
a second receptacle comprising a replacement nucleic acid molecule comprising a coding sequence replacement (CDSR) construct designed for serving as a template for homology-directed repair (HDR) triggered by the DSB, the CDSR construct comprising:
   a. a left homology arm (LHA) comprising a sequence essentially identical to a sequence directly upstream of the gene portion sequence;
   b. a right homology arm (RHA) comprising a sequence essentially identical to a sequence directly downstream of the gene portion sequence; and
   c. the transgene sequence comprising an edited sequence and positioned between the LHA and the RHA, and
instructions for use.

It is appreciated that the kit is designed for the same purpose as the systems described herein. Accordingly, the definitions of terms and embodiments disclosed with reference to the system also apply to the kit.

### List of Sequences for Element Used in the CDSR and CSI Constructs:

### Promoters, Reporters, 3′ Elements:

SFFV: SEQ ID NO: 3
GFP: SEQ ID NO: 4 (corresponding amino acid SEQ ID NO: 68)
BGH: SEQ ID NO: 5
WPRE: SEQ ID NO: 6
PGK: SEQ ID NO: 7
tNGFR: SEQ ID NO: 8 (corresponding amino acid SEQ ID NO: 69)
T2A: SEQ ID NO: 9

### RAG-Specific Sequences

RAG2 LHA-400: SEQ ID NO: 10
TCCAAAGCTACACATCCCAGAGAGAACGGATTCTTGGGAAATGTGGTTCTTTCAGCTGACGCAT
GGTGACTCTTTACGGAAAAGGACTACAATTCCCAGAAATCCTAGGGAGCATATAGTCCGTGGCT
AAAACATGTCCCAGCTCCTTGGATGGAATGGCAGTAAAGGTTCTGTGGCTCTTTACTGACCTAA
CTCCTTGGATTTTCCTCATTCAGTCCCACTGCAAGCGTGTGGGAGGACTTAAAAAAATGCTATT
CACATGTGAAGGAATCTAAATACGATGATTATATGAAGCGATCTCTAAGTCATTTTATTTTATA
ATTCTTTCAGACAAAAATCTACGTACCATCAGAAACTATGTCTCTGCAGATGGTAACAGTCAGT
AATAACATAGCCTTAA
RAG2 RHA-400 (CSI): SEQ ID NO: 11
TTCAGCCAGGCTTCTCACTGATGAATTTTGATGGACAAGTTTTCTTCTTTGGACAAAAAGGCTG
GCCCAAAAGATCCTGCCCCACTGGAGTTTTCCATCTGGATGTAAAGCATAACCATGTCAAACTG
AAGCCTACAATTTTCTCTAAGGATTCCTGCTACCTCCCTCCTCTTCGCTACCCAGCCACTTGCA
CATTCAAAGGCAGCTTGGAGTCTGAAAAGCATCAATACATCATCCATGGAGGGAAAACACCAAA
CAATGAGGTTTCAGATAAGATTTATGTCATGTCTATTGTTTGCAAGAACAACAAAAAGGTTACT
TTTCGCTGCACAGAGAAAGACTTGGTAGGAGATGTTCCTGAAGCCAGATATGGTCATTCCATTA
ATGTGGTGTACAGCCG
RAG2 RHA-400 (CDSR): SEQ ID NO: 12
TTTTGCAAAAGCCTTTCAGATTCAGGTGTATGGAATTTTTGAATCTATTTTTAAAATCATAACA
TTGATTTTAAAAATACATTTTTGTTTATTTAAAATGCCTATGTTTTCTTTTAGTTACATGAATT
AAGGGCCAGAAAAAAGTGTTTATAATGCAATGATAAATAAAGTCATTCTAGACCCTATACATTT
TGAAAATATTTTACCCAAATACTCAATTTACTAATTTATTCTTCACTGAGGATTTCTGATCTGA
TTTTTTATTCAACAAACCTTAAACACCCAGAAGCAGTAATAATCATCGAGGTATGTTTATATTT
ATTATATAAGTCTTGGTAACAAATAACCTATAAAGTGTTTATGACAAATTTAGCCAATAAAGAA
ATTAACACCCAAAAGA
RAG2 RHA-800 (CDSR): SEQ ID NO: 13 =
SEQ ID NO: 12 +
ATTAAATTGATTATTTTGTGCAACATAACAATTCGGCAGTTGGCCAAAACTTAAAAGCA
AGATCTACTACATCCCACATTAGTGTTCTTTATATACCTTCAAGCAACCCTTTGGATTA
TGCCCATGAACAAGTTAGTTTCTCATAGCTTTACAGATGTAGATATAAATATAAATATA
TGTATACATATAGATAGATAATGTTCTCCACTGACACAAAAGAAGAAATAAATAATCTA
CATCAAGTTTGACATGTTTTCCTGAATTACTTGTATGCATTTTCTTTAAGGATTTCCCC
TCCCCATTATATGATGCATGAATGATGTGCCAAAGTCAGTCATTTGAACTAATTATTTG
TGAGAATTTTCATGGAAAACTGGAGCCCATTGCTACTTAGTACTCT
RAG2 RHA-1600 (CDSR): SEQ ID NO: 14 =
SEQ ID NO: 13 +
GGAAAAAGGAAGTGAGTAAGAATACAATAAGAAAGGACCACCAAATAAATCTCATGGAGAAGAA
TAATATTATTTCCCAAAATAATGTAAGTTTAAAGAATTTCATGACATTTACAGGCAGAAGATTT
AAATGGAGGAAAAGAAATGTATTTGTGTGAGTGTGTGTGTGTGTGTGTGTGTGTGTGTAGTTAA
CATGTGGAGATTGCTGCCAGTGAATATTATAAAATCAGTGATCTTGCCAAGGTCTAATTAGACA
CTCGCTAGGATGAAACTAGTTAAAATGACTGTTAATTTTAAGGGTTCAAGACATCAGTTGATAA
CTAGATGACCTTAGAAACAAATGTCTTTCCTCCTGAAATATTTTCCGGAAAAAAAAATTTTCTG
GAAAAACCTTATCTTAAGAGCTTCAGCCACAGTTACAGTGAAGACTCTTACTCCTCACTGAAAG
TCTACAGTGTGTAAGGTACAACTAACAAATTTACGGGAAACATGAATTATGCAAGAGATGAAAC
GCTGGAGTACAAGTTCTTCTGCTGAAAGTTCCATGTCCCCAGATACTGAAATTAACTTTGATAA
ACTGAATCATATTCACTCTGTTTCAATAATGTTGCTCGCTGAATATTCCTGTTGAACAATTGTT
GCACATTGATTTGATCTTTTGTTGTTTCCATCACCTATATAAAAATAATAAAATATAAAAATCT
TCTATTTATATTGATTTACTCTAATTATTAAGATATGTTACCTAAATAACGGCAAAATTAAACT
AATTTTTTCAGGATCACTCCACTTTGATTTTT
RAG1 LHA-800: SEQ ID NO: 15
TGAATTTTTCATCTTAAAAGTCCCTTAGAATCTCAGTCTATGTACACTCAGGTTTGTTGCAGGT
TTAGAGTTCCGTGTTTTTTGTTTCTAATGTAGACACAGCCTTATAATTTACAACAGCATTCACT
AATTAAAATTGTAAGCATAATTACTATCCACGATACTTATTATTAGTTTGCATTCATAAAGCTC
AAAATTCACTTCATCCTTTCAAGTAGTGAATAATTAGTTTCTTTGGGTTTGCAGCTTTATCATC
CTTTTATGACCCATTTGGAAGAAATAAACAACCAACCCCCTGGAAGACTGCTTTAAAAAGCTGG
AAATACATTGTCCAGCTAGTACAATGAGGCTAATACAATGTGGAAAATATTACTTTTCTTTGAT
TTTAGTAGCCTGTTTATCTTTACATTTACTGAACAAATAACTATTGAGCACCTAATGTATACTG
GGACCCTTGGGGAGGCAAAGATGAATCAAAGATTCTGTCCTTAAAGACCTTAAGGTTTTTGTGG
AAGGAAATAAAACTTTACATGTATATATTTAAGCACTTATATGTGTGTAACAGGTATAAGTAAC
CATAAACACTGTCAGAAGAGGAAATAACTCTATGATCAGCACCTAACATGATATATTAAGGTAG
AAGATTTAATACATATCTTTTGGAATACATGAATAAATAATTGAATGTATTTATTTTTATTATT
TATAAGATACATCAGTGGGATATTGATATTGGTCTTAATATGACTTGTTTTCATTGTTCTCAGG
TACCTCAGCCAGCATGGCAGCCTCTTTCCCACC
RAG1 RHA-800 (CSI): SEQ ID NO: 16
CACCTTGGGACTCAGTTCTGCCCCAGATGAAATTCAGCACCCACATATTAAATTTTCAGAATGG
AAATTTAAGCTGTTCCGGGTGAGATCCTTTGAAAAGACACCTGAAGAAGCTCAAAAGGAAAAGA
AGGATTCCTTTGAGGGGAAACCCTCTCTGGAGCAATCTCCAGCAGTCCTGGACAAGGCTGATGG
TCAGAAGCCAGTCCCAACTCAGCCATTGTTAAAAGCCCACCCTAAGTTTTCAAAGAAATTTCAC
GACAACGAGAAAGCAAGAGGCAAAGCGATCCATCAAGCCAACCTTCGACATCTCTGCCGCATCT
GTGGGAATTCTTTTAGAGCTGATGAGCACAACAGGAGATATCCAGTCCATGGTCCTGTGGATGG
TAAAACCCTAGGCCTTTTACGAAAGAAGGAAAAGAGAGCTACTTCCTGGCCGGACCTCATTGCC
AAGGTTTTCCGGATCGATGTGAAGGCAGATGTTGACTCGATCCACCCCACTGAGTTCTGCCATA
ACTGCTGGAGCATCATGCACAGGAAGTTTAGCAGTGCCCCATGTGAGGTTTACTTCCCGAGGAA
CGTGACCATGGAGTGGCACCCCCACACACCATCCTGTGACATCTGCAACACTGCCCGTCGGGGA
CTCAAGAGGAAGAGTCTTCAGCCAAACTTGCAGCTCAGCAAAAAACTCAAAACTGTGCTTGACC
AAGCAAGACAAGCCCGTCAGCACAAGAGAAGAGCTCAGGCAAGGATCAGCAGCAAGGATGTCAT
GAAGAAGATCGCCAACTGCAGTAAGATACATCT
RAG1 RHA-800 (CDSR): SEQ ID NO: 17
GTAGGGCAACCACTTATGAGTTGGTTTTTGCAATTGAGTTTCCCTCTGGGTTGCATTGAGGGCT
TCTCCTAGCACCCTTTACTGCTGTGTATGGGGCTTCACCATCCAAGAGGTGGTAGGTTGGAGTA
AGATGCTACAGATGCTCTCAAGTCAGGAATAGAAACTGATGAGCTGATTGCTTGAGGCTTTTAG
TGAGTTCCGAAAAGCAACAGGAAAAATCAGTTATCTGAAAGCTCAGTAACTCAGAACAGGAGTA
ACTGCAGGGGACCAGAGATGAGCAAAGATCTGTGTGTGTTGGGGAGCTGTCATGTAAATCAAAG
CCAAGGTTGTCAAAGAACAGCCAGTGAGGCCAGGAAAGAAATTGGTCTTGTGGTTTTCATTTTT
TTCCCCCTTGATTGATTATATTTTGTATTGAGATATGATAAGTGCCTTCTATTTCATTTTTGAA
TAATTCTTCATTTTTATAATTTTACATATCTTGGCTTGCTATATAAGATTCAAAAGAGCTTTTT
AAATTTTTCTAATAATATCTTACATTTGTACAGCATGATGACCTTTACAAAGTGCTCTCAATGC
ATTTACCCATTCGTTATATAAATATGTTACATCAGGACAACTTTGAGAAAATCAGTCCTTTTTT
ATGTTTAAATTATGTATCTATTGTAACCTTCAGAGTTTAGGAGGTCATCTGCTGTCATGGATTT
TTCAATAATGAATTTAGAATACACCTGTTAGCTACAGTTAGTTATTAAATCTTCTGATAATATA
TGTTTACTTAGCTATCAGAAGCCAAGTATGAT
RAG1 RHA-1600 (CDSR): SEQ ID NO: 18 =
SEQ ID NO: 17 +
TCTTTATTTTTACTTTTTCATTTCAAGAAATTTAGAGTTTCCAAATTTAGAGCTTCTGCATACA
GTCTTAAAGCCACAGAGGCTTGTAAAAATATAGGTTAGCTTGATGTCTAAAAATATATTTCATG
TCTTACTGAAACATTTTGCCAGACTTTCTCCAAATGAAACCTGAATCAATTTTTCTAAATCTAG
GTTTCATAGAGTCCTCTCCTCTGCAATGTGTTATTCTTTCTATAATGATCAGTTTACTTTCAGT
GGATTCAGAATTGTGTAGCAGGATAACCTTGTATTTTTCCATCCGCTAAGTTTAGATGGAGTCC
AAACGCAGTACAGCAGAAGAGTTAACATTTACACAGTGCTTTTTACCACTGTGGAATGTTTTCA
CACTCATTTTTCCTTACAACAATTCTGAGGAGTAGGTGTTGTTATTATCTCCATTTGATGGGGG
TTTAAATGATTTGCTCAAAGTCATTTAGGGGTAATAAATACTTGGCTTGGAAATTTAACACAGT
CCTTTTGTCTCCAAAGCCCTTCTTCTTTCCACCACAAATTAATCACTATGTTTATAAGGTAGTA
TCAGAATTTTTTTAGGATTCACAACTAATCACTATAGCACATGACCTTGGGATTACATTTTTAT
GGGGCAGGGGTAAGCAAGTTTTTAAATCATTTGTGTGCTCTGGCTCTTTTGATAGAAGAAAGCA
ACACAAAAGCTCCAAAGGGCCCCCTAACCCTCTTGTGGCTCCAGTTATTTGGAAACTATGATCT
GCATCCTTAGGAATCTGGGATTTGCCAGTTGC
RAG1 RHA-2000 (CDSR): SEQ ID NO: 19 =
SEQ ID NO: 18 +
TGGCAATGTAGAGCAGGCATGGAATTTTATATGCTAGTGAGTCATAATGATATGTTAGTGTTAA
TTAGTTTTTTCTTCCTTTGATTTTATTGGCCATAATTGCTACTCTTCATACACAGTATATCAAA
GAGCTTGATAATTTAGTTGTCAAAAGTGCATCGGCGACATTATCTTTAATTGTATGTATTTGGT
GCTTCTTCAGGGATTGAACTCAGTATCTTTCATTAAAAAACACAGCAGTTTTCCTTGCTTTTTA
TATGCAGAATATCAAAGTCATTTCTAATTTAGTTGTCAAAAACATATACATATTTTAACATTAG
TTTTTTTGAAAACTCTTGGTTTTGTTTTTTTGGAAATGAGTGGGCCACTAAGCCACACTTTCCC
TTCATCCTGCTTAATC
dcoRAG2 cDNA: SEQ ID NO: 20, corresponding amino
acid SEQ ID NO: 67 (nu 3-1538))
TCCAGCCCGGCTTCAGCCTCATGAACTTCGACGGCCAGGTGTTTTTTTTCGGCCAGAAGGGATG
GCCTAAGAGGAGCTGTCCTACCGGCGTGTTCCACCTCGACGTGAAGCACAATCACGTGAAGCTC
AAACCCACCATCTTTAGCAAAGACAGCTGTTATCTGCCCCCCCTGAGATATCCCGCTACCTGTA
CCTTTAAGGGATCCCTGGAAAGCGAGAAACACCAGTATATTATTCACGGCGGCAAGACCCCCAA
TAACGAAGTGAGCGACAAAATCTACGTGATGAGCATCGTGTGTAAAAATAATAAGAAAGTGACC
TTCAGATGTACCGAAAAGGATCTGGTGGGCGACGTGCCCGAGGCTAGGTACGGCCACAGCATCA
ACGTCGTCTATTCCAGAGGCAAGAGCATGGGCGTGCTGTTCGGCGGCAGAAGCTATATGCCCAG
CACACATAGGACAACCGAGAAGTGGAACAGCGTGGCCGATTGTCTCCCTTGCGTGTTTCTCGTC
GACTTCGAGTTCGGCTGCGCCACCAGCTATATCCTGCCCGAGCTGCAAGACGGCCTGAGCTTCC
ACGTGAGCATCGCTAAGAACGATACAATTTACATCCTGGGCGGCCACAGCCTGGCTAACAACAT
TAGACCCGCTAATCTCTATAGGATCAGAGTGGACCTGCCTCTCGGCTCCCCCGCCGTCAACTGT
ACCGTGCTGCCCGGCGGCATTAGCGTGAGCAGCGCCATTCTCACCCAGACCAATAACGACGAGT
TCGTGATCGTGGGCGGATACCAACTGGAGAACCAGAAGAGGATGATTTGTAATATTATTAGCCT
GGAAGATAATAAAATCGAGATCAGAGAAATGGAAACACCCGACTGGACACCCGATATCAAACAT
TCCAAAATCTGGTTCGGCTCCAATATGGGCAACGGCACCGTGTTCCTGGGAATCCCCGGCGATA
ACAAGCAGGTGGTGAGCGAGGGCTTTTACTTTTACATGCTGAAGTGCGCCGAGGACGACACCAA
CGAGGAACAAACCACCTTTACCAATAGCCAGACCAGCACCGAGGACCCCGGCGACAGCACCCCT
TTCGAGGATAGCGAGGAGTTCTGCTTTAGCGCCGAGGCCAACAGCTTCGACGGCGACGACGAGT
TCGATACATACAACGAGGACGACGAGGAGGACGAAAGCGAAACCGGATATTGGATCACCTGTTG
TCCCACCTGCGACGTCGACATTAATACCTGGGTGCCCTTTTACAGCACCGAACTGAATAAGCCT
GCTATGATTTATTGTAGCCACGGCGACGGCCATTGGGTGCACGCCCAATGTATGGACCTCGCCG
AGAGAACCCTGATTCACCTCAGCGCCGGCTCCAATAAATACTATTGTAACGAACACGTCGAAAT
CGCCAGGGCCCTGCATACCCCTCAGAGGGTGCTGCCTCTGAAGAAACCCCCCATGAAGAGCCTG
AGAAAGAAGGGCAGCGGCAAGATTCTGACCCCCGCTAAAAAGAGCTTCCTGAGGAGACTGTTCG
AC
RAG2 endogenous 3′ UTR: SEQ ID NO: 21
TTTTGCAAAAGCCTTTCAGATTCAGGTGTATGGAATTTTTGAATCTATTTTTAAAATCATAACA
TTGATTTTAAAAATACATTTTTGTTTATTTAAAATGCCTATGTTTTCTTTTAGTTACATGAATT
AAGGGCCAGAAAAAAGTGTTTATAATGCAATGATAAATAAAGTCATTCTAGACCCTATACATTT
TGAAAATATTTTACCCAAATACTCAATTTACTAATTTATTCTTCACTGAGGATTTCTGATCTGA
TTTTTTATTCAACAAACCTTAAACACCCAGAAGCAGTAATAATCATCGAGGTATGTTTATATTT
ATTATATAAGTCTTGGTAACAAATAACCTATAAAGTGTTTATGACAAATTTAGCCAATAAAGAA
ATTAACACCCAAAAGAATTAAATTGATTATTTTGTGCAACATAACAATTCGGCAGTTGGCCAAA
ACTTAAAAGCAAGATCTACTACATCCCACATTAGTGTTCTTTATATACCTTCAAGCAACCCTTT
GGATTATGCCCATGAACAAGTTAGTTTCTCATAGCTTTACAGATGTAGATATAAATATAAATAT
ATGTATACATATAGATAGATAATGTTCTCCACTGACACAAAAGAAGAAATAAATAATCTACATC

### List of Constructs

### RAG2 KO Constructs:

CSI_GFP-BGHpA_400×400 (SEQ ID NO: 22): RAG2 LHA-400, SFFV, GFP, BGH, RAG2 RHA-400 (CSI); corresponding amino acid SEQ ID NO: 70 (GFP, nu 946-1626)
CDSR_GFP-BGHpA_400×400 (SEQ ID NO: 23): RAG2 LHA-400, SFFV, GFP, BGH, RAG2 RHA-400 (CDSR); corresponding amino acid SEQ ID NO: 71 (GFP, nu 946-1626)
CDSR_GFP-BGHpA_400×800 (SEQ ID NO: 24): RAG2 LHA-400, SFFV, GFP, BGH, RAG2 RHA-800 (CDSR); corresponding amino acid SEQ ID NO: 72 (GFP, nu 946-1626)
CDSR_GFP-BGHpA_400×1600 (SEQ ID NO: 25): RAG2 LHA-400, SFFV, GFP, BGH, RAG2 RHA-1600 (CDSR); corresponding amino acid SEQ ID NO: 73 (GFP, nu 946-1626)
CDSR_GFP-NoBGHpA_400×1600 (SEQ ID NO: 26): RAG2 LHA-400, SFFV, GFP, RAG2 RHA-1600 (CDSR); corresponding amino acid SEQ ID NO: 74 (GFP, nu 946-1626)
CDSR_GFP-WPRE-BGHpA_400×1600 (SEQ ID NO: 27): RAG2 LHA-400, SFFV, GFP, WPRE; BGH; RAG2 RHA-1600 (CDSR); corresponding amino acid SEQ ID NO: 75 (GFP, nu 946-1626)

### RAG2 KI Constructs

CSI_Corr (SEQ ID NO: 28): RAG2 LHA-400, dcoRAG2 cDNA, RAG2 endogenous 3′ UTR, PGK, tNGFR, BGH, RAG2 RHA-400 (CSI); corresponding amino acid SEQ ID NO: 80 (dcoRAG2, nu 358-1941), SEQ ID NO: 81 (tNGFR, nu 3103-3945)
CDSR_Corr_Endo3′UTR (SEQ ID NO: 29): RAG2 LHA-400, dcoRAG2 cDNA, T2A, tNGFR, RAG2 RHA-800 (CDSR); corresponding amino acid SEQ ID NO: 82 (dcoRAG2+T2A+tNFGR, nu 358-2844)
CDSR_Corr_BGHpA (SEQ ID NO: 30): RAG2 LHA-400, dcoRAG2 cDNA, T2A, tNGFR, BGH, RAG2 RHA-800 (CDSR); corresponding amino acid SEQ ID NO: 83 (dcoRAG2+T2A+tNFGR, nu 358-2844)
CDSR_Corr_WPRE-BGHpA (SEQ ID NO: 31): RAG2 LHA-400, dcoRAG2 cDNA, T2A, tNGFR, WPRE, BGH, RAG2 RHA-800 (CDSR); corresponding amino acid SEQ ID NO: 84 (dcoRAG2+T2A+tNFGR, nu 358-2844)

### RAG1 KO Constructs:

RAG1_CSI_GFP-BGHpA_800×800 (SEQ ID NO: 32): RAG1 LHA-800, SFFV, GFP, BGH, RAG1 RHA-800 (CSI); corresponding amino acid SEQ ID NO: 76 (GFP, nu 1331-2011)
RAG1_CDSR_GFP-BGHpA_800×800 (SEQ ID NO: 33): RAG1 LHA-800, SFFV, GFP, BGH, RAG1 RHA-800 (CDSR); corresponding amino acid SEQ ID NO: 77 (GFP, nu 1331-2011)
RAG1_CDSR_GFP-BGHpA_800×1600 (SEQ ID NO: 34): RAG1 LHA-800, SFFV, GFP, BGH, RAG1 RHA-1600 (CDSR); corresponding amino acid SEQ ID NO: 78 (GFP, nu 1331-2011)
RAG1_CDSR_GFP-BGHpA_800×2000 (SEQ ID NO: 35): RAG1 LHA-800, SFFV, GFP, BGH, RAG1 RHA-2000 (CDSR); corresponding amino acid SEQ ID NO: 79 (GFP, nu 1331-2011)

For the purpose of the present application, unless indicated otherwise, when referring to double stranded sequences (such as gene, gene portion sequence, CDSR, etc.), “5′” is generally used to refer to the upstream side of the sequence, and “3′” is generally used to refer to the downstream side of the sequence. The “upstream” and “downstream” annotations are with reference to the direction of transcription of the respective gene, i.e.—“upstream” is in the direction of the promoter while “downstream” is in the direction of the polyA sequence.

The term “sequence”, when used without specifying whether it is a nucleic acid or an amino acid sequence, is interpreted according to context. For example, when it is part of a gene or genomic sequence, then it is a nucleic acid sequence, and when it is part of a gene product that is a protein, it is an amino acid sequence.

The term “gene product”, as used herein, may refer to an RNA product or an amino acid product. However, if the gene includes a coding sequence, then the gene product relates to a protein or an amino acid product. Also, in some cases it may be understood from the context that the gene product is an amino acid sequence rather than an RNA sequence.

The term “essentially identical”, used with reference to the level of identity between sequences, means that the sequences are meant to be identical. However, due to natural variations, there may unintentionally be variation at a low level and therefore the sequences may not be 100% identical. These variations should not affect the result and should therefore not be important for the invention. It is generally estimated that the level of identity for essentially identical sequences is above 95%, 96%, 97%, 98%, or 99%.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical objects of the article. By way of example, “an element” means one element or more than one element.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

While certain embodiments of the invention have been illustrated and described, it will be clear that the invention is not limited to the embodiments described herein. Numerous modifications, changes, variations, substitutions, and equivalents will be apparent to those skilled in the art without departing from the spirit and scope of the present invention as described by the claims, which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Materials and Methods

### Cells and Cell-Culture Conditions

Cord blood (CB)-derived CD34⁺ hematopoietic stem and progenitor cells (HSPCs) were obtained from Sheba Medical Center CB bank under Institutional Review Board-approved protocols. CD34⁺ HSPCs were isolated via magnetic bead-separation (Miltenyi Biotec). CD34⁺ HSPCs were cultured in SFEM II enriched with 100 ng/ml Flt3-Ligand, 100 ng/ml TPO, 100 ng/ml SCF, 0.035 mg/μl UM171, 0.75 mg/μl SR1 (TEMCELL Technologies, Inc.), 20 unit/ml penicillin, and 20 mg/ml streptomycin (Biological Industries, Beit Haemek, Israel) at 37° C., 5% CO₂, and 5% O₂. Each repeat in this paper was performed on biologically unique CD34⁺ HSPCs from different CB donors.

### CRISPR-Cas9 Preparation and Nucleofection

CRISPR-Cas9 RNP complex preparation and nucleofection were conducted in accordance with the extensive protocol published in Shapiro et al. (Shapiro et al., Chemical Modification of Guide RNAs for Improved CRISPR Activity in CD34+ Human Hematopoietic Stem and Progenitor Cells. Methods Mol Biol. 2162:37-48). A single guide RNA (sgRNA, Alt-R® sgRNA, IDT) with 2′-O-methyl-3′-phosphorothioate (MS) end chemical modifications was complexed with Cas9 at a molar ratio of 1:2.5 (Cas9:sgRNA) for 10-20 min at 25° C. to form the RNP complex. The RAG2 sgRNA variable region sequence was 5′-UGAGAAGCCUGGCUGAAUUA-3′ SEQ ID NO: 1). RNP complexes were added to CD34⁺ HSPCs reconstituted in P3 Primary Cell electroporation solution, according to the manufacturer's protocol (Lonza) at a final molar concentration of 4 μM. The cell solution was electroporated in the Lonza 4D-Nucleofector using the DZ-100 program.

rAAV6-Based DNA Construct Design and Production

All rAAV6 vector-based plasmids were cloned using NEBuilder HiFi DNA Assembly Master Mix (cat. no. E2621L, New England Biolabs [NEB]) into the pAAV-MCS plasmid (Agilent Technologies) containing AAV2-specific inverted terminal repeats (ITRs), as described by Iancu et al. (supra). The pDGM6 plasmid containing the AAV6 cap genes, AAV2 rep genes, and adenovirus helper genes, was a gift from David Russell (University of Washington). The final rAAV6 constructs (further detailed below) were produced by co-introducing the two plasmids into HEK293 cells, and harvesting rAAV6 constructs to be used for genome targeting, as described by Grieger et al. 2006, Nat. Protoc. 1(3):1412-1428, and Khan et al, 2011, Nat. Protoc. 6(4):482-501.

### Genome Targeting and Quantification

After electroporation with the RAG2 RNP complex, CD34⁺ HSPCs were seeded at a density of 0.4×10⁶ cells/ml for 24 hrs. Following the incubation period, the cell density was adjusted to 0.25×10⁶ cells/ml for an additional 24 hrs. In homology directed repair (HDR) experiments, the cells were transduced with the rAAV6 construct at MOIs of either 12500 or 25000 VG/cell within 5 min of electroporation. Cells were cultured at 37° C., 5% CO₂, and 5% O₂ in StemSpan™ SFEM II enriched medium as noted above. Flow cytometry analyses were performed on the BD LSRFortessa™ (BD Biosciences), Aria III cell sorter (BD Biosciences), or the Accuri C6 flow cytometer (BD Biosciences).

### In Vitro T Cell Differentiation (IVTD) System and Immunostaining

CD34⁺ HSPCs were cultured in the StemSpan™ T Cell Generation Kit (STEMCELL Technologies, Inc.). For the first 14 days, cells were cultured in StemSpan™ SFEM II medium containing Lymphoid Progenitor Expansion Supplement in plates pre-coated with the Lymphoid Differentiation Coating Material. Cells were then harvested and re-seeded for an additional 14 days in StemSpan™ SFEM II medium containing the T Cell Progenitor Maturation Supplement on pre-coated plates. Flow cytometry analysis was conducted on days 14 and 28 of IVTD using the LSR Fortessa™ (BD Biosciences) and T cell marker antibody cocktails. Antibody staining: cells were stained with PE/Cy7-anti-CD7 (clone: CD7-6B7, BioLegend), BV421-anti-CD5 (clone: UCHT2, BioLegend), PE-anti-CD1a (clone: BL6, Beckman Coulter), and APC-anti-NGFR (clone: ME20.4, BioLegend) antibodies. On day 28 of IVTD, cells were stained with PE/Cy7-anti-CD4 (clone: RPA-T4, BioLegend), APC-r700-anti-CD8a (clone: RPA-T8, BD Horizon™) BV421-anti-CD3 (clone: UCHT1, BioLegend), and APC-anti-NGFR (clone: ME20.4, BioLegend) antibodies. BD Horizon™ Fixable Viability Stain 510 was performed on all collected cells at both time points. Gating strategies were based on fluorescence minus one (FMO) plus isotype control samples using the following isotypes: PE/Cy7 Mouse IgG2a κ, (BioLegend), BV421 Mouse IgG1 κ (BioLegend), PE Mouse IgG1 κ (BioLegend), PE/Cy7 Mouse IgG1 κ (BioLegend), APC-r700 Mouse IgG1 κ (BD Biosciences), and APC Mouse IgG1 κ (BioLegend).

Digital DropletPCR™ (ddPCR™)

Genomic integration quantification for HDR experiments was performed by Digital Droplet PCR™ (ddPCR™, Bio-Rad). DNA was extracted from cell populations using GeneJET Genomic DNA Purification Kit (Thermo Fisher Scientific). Each ddPCR reaction contained a HEX reference assay detecting the CCRL2 gene to quantify the chromosome 3 copy number input (Gomez-Ospina et al., Human genome-edited hematopoietic stem cells phenotypically correct mucopolysaccharidosis type I, Nat Commun. 10(1))). FAM assays (PrimeTime® Standard qPCR Assay (IDT)) for either KO (disruption) or KI (correction) constructs were designed to detect the locus-specific vector integration. The ddPCR reaction was carried with the following reagents: 10 μl of ddPCR Supermix for Probes No dUTP (Bio-Rad), 1 μl each of FAM and HEX PrimeTime® Standard qPCR Assay (IDT), 1 μl restriction enzyme mix (5 μl EcoRI-HF® (NEB), 2 μl nuclease-free water, 1 μl CutSmart Buffer 10× (NEB)), genomic template DNA, and supplemented to a total of 20 μl with nuclease-free water. Droplet samples were prepared according to the manufacturer's protocol (Bio-Rad) and 40 μl of the droplet output was transferred to a 96-well plate and amplified in a Bio-Rad PCR thermocycler (Bio-Rad) at the following PCR conditions: 1 cycle at 95° C. for 10 min, then 40 cycles of 95° C. for 30 sec and 55° C. for 3 min, followed by 1 cycle at 98° C. for 10 min with a ramp rate of 2.2° C./sec. Following the PCR, the plate was read in the QX200 Droplet Reader (Bio-Rad) and the data was analyzed using the QuantaSoft analysis software (Bio-Rad). Primer and probe sequences are listed in Table 1.

mRNA Quantification

RNA was extracted using Direct-zol™ RNA Miniprep Plus (Zymo Research) from differentiated T cells obtained on day 28 of IVTD. cDNA preparation was executed from RNA, using Oligo d(T)23 VN-S1327S (NEB), dNTPs 10 mM (Sigma-Aldrich), and M-MuLV Reverse Transcriptase (NEB). qRT-PCR reactions were conducted using the TaqMan® Fast Advanced Master Mix (Thermo Fisher Scientific) in the StepOnePlus™ Real-Time PCR System (Thermo Fisher Scientific). PCR conditions were as follows: uracil-N-glycosylase gene (UNG) incubation at 50° C. for 2 min, polymerase activation at 95° C. for 20 sec, and 40 cycles at 95° C. for 1 sec and at 60° C. for 20 sec. Primer and probe sequences are listed in Table 1.

### TRB and TRG V(D)J Assessment

Genomic DNA was extracted using the GeneJET Genomic DNA Purification Kit (Thermo Fisher Scientific) from differentiated T cells on day 28 of IVTD. For TRG assessment via PCR amplification, 12 possible CDR3 clones were amplified using combinations of 4 primers for the Vγ regions and 3 primers for the Jγ regions (primer sequences are presented in Table 2). The PCR products were run on a 2% agarose gel. For deep sequencing of the TRB and TRG repertoires, the same genomic DNA was amplified using a multiplex master mix from either the LymphoTrack® TRB assay and/or LymphoTrack® TRG assay kits (Invivoscribe, Inc.). The amplicons were purified and sequenced using the MiSeq V2 (500 cycles) kit, with 250 bp paired-end reads (Illumina). The resulting FASTQ files were analyzed by the LymphoTrack Software (Invivoscribe, Inc.) and by the IMGT® Software (The International ImMunoGeneTics Information System®, HighV-QUEST, http://www.imgt.org). The analysis of the incidence and clonality of TRB and TRG rearrangement sequences was performed for visual representation by the TreeMap Software (Macrofocus GmbH). Unique CDR3 sequence and length were determined from the total productive sequences. Lastly, the Shannon's H and Simpson's 1-D diversity indices were calculated using the PAST Software (Hammer et al., Past: Paleontological Statistics Software Package for Education and Data Analysis, Palaeontologia Electronica. 4(1):178).

### Data Sharing Statement

The TRB and TRG sequencing data as well as the ITR-seq and ONT long-read sequencing data were deposited to the Sequence Read Archive (SRA) at the National Center for Biotechnology Information (NCBI), under accession number: PRJNA926613.

TABLE 1
droplet digital PCR (ddPCR) and qRT-PCR assay sequences

| | | |
|---|---|---|
| SEQ | | |
| ID | Name | Sequence |

DNA primers/probes

| | | | |
|---|---|---|---|
| 36 | RAG2 KO | F | CTCTCCCAGAGCAACAAAGAC |
| 37 | | R | TTTCCATGCCTTGCAAAATGG |
| 38 | | P | 56-FAM/CCTACAGGT/ZEN/GGGGTCTTTCATTCC/3IABkFQ |
| 39 | RAG2 KI | F | TCTCCCAGAGCAACAAAG |
| 40 | | R | GAAGTTCATGAGGCTGAAG |
| 41 | | P | 56-FAM/CATAGCCTT/ZEN/AATCCAGCCCG/3IABkFQ |
| 42 | RAG1 KO | F | CAGTGACTTTCAGGATGACCT |
| 43 | | R | CAGCTAGCTTGCCAAACC |
| 44 | | P | 56-FAM/CCATGCTGG/ZEN/CTGAGGTACC/3IABkFQ |
| 45 | CCRL2 | F | GCTGTATGAATCCAGGTCC |
| 46 | | R | CCTCCTGGCTGAGAAAAAG |
| 47 | | P | 5HEX/TGTTTCCTC/ZEN/CAGGATAAGGCAGCTGT /3IABkFQ |

RNA primers/probes

| | | | |
|---|---|---|---|
| 48 | RAG2 | F | CCAAGTGCTGACAATTAATACCTG |
| 49 | Endo. | R | GACATGGTTATGCTTTACATCCAG |
| 50 | | P | 56-FAM/CATCAGTGA/ZEN/GAAGCCTGGCTGAATTAAGG |
| | | | /3IABkFQ |
| 51 | dcoRAG2 | F | CAAGTGCTGACAATTAATACCT |
| 52 | CDNA | R | GAAGTTCATGAGGCTGAAG |
| 53 | | P | 56-FAM/CATAGCCTT/ZEN/AATCCAGCCCG/3IABkFQ |
| 54 | RAG2 | F | CCAAGTGCTGACAATTAATACCTG |
| 55 | comp | R | GACATAGTTTCTGATGGTACGTAGA |
| 56 | | P | 56-FAM/TCACGCCTC/ZEN/TCTGAATCTTTGCCG/3IABkFQ |
| 57 | GAPDH | F | ACATCGCTCAGACACCATG |
| 58 | | R | TGTAGTTGAGGTCAATGAAGGG |
| 59 | | P | 56-FAM/ |
| | | | AAGGTCGGA/ZEN/GTCAACGGATTTGGTC/3IABkFQ |

F: forward primer; R: reverse primer; P: probe; Endo: endogenous; comp: comparison.
The probes were modified by a fluorescent tag (56-FAM) at the 5′-end and a quencher (3IABKFQ) at the 3′- end, and an internal and 3′ quencher ZEN in the sequence (IDT).

TABLE 2
TRG PCR amplification primers for
V(D)J recombination assessment

| SEQ | | |
|---|---|---|
| ID | Region | Sequence |
| 60 | Jγ_1/2 | TACCTGTGACAACCAGTGTTG |
| 61 | Jγ_P | ACTTACCTGTAATGATAAGCTTT |
| 62 | Jγ_P1/2 | TTACCAGGCGAAGTTACTATG |
| 63 | Vγ_9 2 | ACCTGGTGAAGTCATACAGTTC |
| 64 | Vγ_11 | CTTCCACTTCCACTTTGAAA |
| 65 | Vγ_f1 | ACTGGTACCTACACCAGGAGG |
| 66 | Vγ_10-2 | AGCATGGGTAAGACAAGCAA |

### Example 1: Various Homology Directed Repair (HDR) Strategies: Cut-Site Insertion (CSI) Vs. Coding Sequence (CDS) Replacement (CDSR)

RAG2-SCID is caused by mutations scattered throughout the CDS of the RAG2 gene. Therefore, a universal correction technique that would suit all RAG2-SCID patients requires the delivery of an intact copy of the complete RAG2 CDS. While knock-in (KI) of an intact CDS at the endogenous locus would achieve this, this strategy could interfere with the 3D chromatin architecture and critical endogenous gene regulation by moving regulatory elements further downstream from the transgene. This could disrupt spatial crosstalk between functional elements upstream and downstream of the RAG2 gene, such as promoter and/or enhancer sequences. Hence, in order to replace the entire coding sequence and preserve upstream and downstream regulatory elements at similar relative positions, the designed construct included a left homology arm (LHA) upstream of the start codon (adjacent to the cut site), and a right homology arm (RHA) distanced from the cut site, downstream of the RAG2 stop codon.

Two rAAV6 constructs were initially constructed for integrating a GFP expression cassette under the regulation of a spleen focus-forming virus (SFFV) promoter and BGHpA sequence after delivery of a RAG2 sgRNA/Cas9 ribonucleoprotein (RNP) complex via electroporation into CD34⁺ HSPCs.

The first construct, a cut-site-insertion construct (CSI_GFP-BGHpA_400×400), uses 400 bp homology arms immediately flanking the Cas9-induced cut site for construct insertion, while the second construct, a CDS-replacement construct, or a CDSR-KO, (CDSR_GFP-BGHpA_400×400), uses a 400 bp LHA spanning the immediate sequence upstream to the Cas9 cut site and a 400 bp RHA spanning the immediate sequence downstream to the RAG2 stop codon, to replace the entire RAG2 CDS with the DNA construct) (FIG. 1A).

Two days post-editing, it was found via flow cytometry that the HDR efficiency of the CSI_GFP-BGHpA_400×400 construct was significantly higher than that of the CDSR_GFP-BGHpA_400×400 construct (21.8% and 9.1%, respectively) (FIG. 1B). Attempting to improve the HDR efficiency of the CDSR technique, two additional rAAV6 constructs were designed, with RHAs extended from 400 bp to 800 bp and 1,600 bp, spanning the immediate region downstream to the RAG2 stop codon (CDSR_GFP-BGHpA_400×800 and CDSR_GFP-BGHpA_400×1600, respectively, FIG. 1A). Extending to 800 bp produced HDR efficiency significantly higher than the CDSR_GFP-BGHpA_400×400 construct (14.8%), and extending to 1,600 bp provided an HDR efficiency comparable to that of the CSI construct observed (25.2%). (FIG. 1B). Using the RAG2-KO F and R primers and RAG2 probe for droplet digital PCR (ddPCR), it was confirmed that the HDR efficiencies as determined by flow cytometry were accurate and locus-specific (FIG. 1C). To validate that the CDSR strategy is broadly applicable and not specific only to the RAG2 locus, a set of rAAV6 constructs was designed to introduce a GFP expression cassette into the RAG1 locus (data not shown). Similar to RAG2, a highly specific RAG1 sgRNA, (SEQ ID NO: 2, UUGACUCAGGGUUCCACCCA) was used, that targeted just downstream from the RAG1 ATG start codon. Since RAG1 CDS is longer than that of RAG2, the CDSR method here replaced 3,112 bp as opposed to only 1541 bp at the RAG2 locus. While highly efficient HDR was demonstrated at the RAG1 locus as well, it was found that longer homology arms were required to do so (LHA of 800 bp and RHA of 800-2000 bp).

Interestingly, a significantly higher mean fluorescence intensity (MFI) of GFP-expressing cells after integration of the CDSR constructs was observed via flow cytometry compared to that of the CSI constructs (FIG. 1D).

### Example 2: Synthetic polyA Sequences and/or Cis-Acting Promoter Response Elements (PREs) Affect Transgene Expression

To modulate transgene expression further, the impact of synthetic 3′ regulatory elements on transgene expression was tested. Thus, two additional CDSR constructs were designed based on the CDSR_GFP-BGHpA_400×1600 construct: CDSR_GFP-WPRE-BGHpA_400×1600, containing both a WPRE and an BGHpA sequence, and CDSR_GFP-NoBGHpA_400×1600, not containing either WPRE or BGHpA elements, thus allowing GFP expression to be controlled by the endogenous RAG2 3′-UTR. (FIG. 2A).

While the CDSR_GFP-BGHpA_400×1600 and CDSR_GFP-WPRE-BGHpA_400×1600 constructs produced comparable HDR efficiencies, the CDSR_GFP-NoBGHpA_400×1600 construct induced a lower rate of HDR as observed by flow cytometry and confirmed by ddPCR as explained above (FIGS. 2B-C). Interestingly, the three constructs produced significantly different MFI levels, with CDSR_GFP-BGHpA_400×1600 (2.8×10⁶) being the highest followed by CDSR_GFP-WPRE-BGHpA_400×1600 and CDSR_GFP-NoBGHpA_400×1600 (1.6×10⁶ and 0.3×10⁶, respectively) (FIG. 2D), highlighting the impact of the synthetic 3′-UTRs in modulating expression patterns.

### Example 3: KI-KO Genotype Engineering in Healthy Donor (HD)-Derived HSPCs Using Two-Part Enrichment Strategy

### Design and Testing of KI CDSR Constructs

Since RAG2 gene regulation is critical, the RAG2-correction strategy suggested by Iancu et al., (supra) was fine-tuned by using the above-described CDSR method. Replacing the entire CDS resulted in transgene expression being driven by the RAG2 endogenous promoter and 3′-UTR, thus enabling the transgenic diverged codon-optimized RAG2 (dcoRAG2) cDNA expression patterns to most similarly resemble that of endogenous RAG2. Additionally, by replacing the entire CDS (˜1.5 kb), as opposed to pushing the sequence ˜4 kb downstream in the case of HDR via insertion, the proximity of the RAG genes was more closely maintained, conserving the ability to form a chromatin hub super enhancer necessary for proper expression. A CDSR correction (CDSR-KI) construct was constructed (CDSR_Corr_Endo3′UTR, FIG. 3A) with a 400×800 bp homology arm pattern (400 bp LHA, 800 bp RHA) for knock-in of the dcoRAG2 cDNA.

To track the expression of dcoRAG2 cDNA and enrich for cells with successful integration, the dcoRAG2 stop codon was eliminated and replaced with a T2A self-cleaving peptide sequence followed by a truncated nerve growth factor receptor (tNGFR) reporter gene, producing in-frame transcription of the two sequences (dcoRAG2 cDNA and tNGFR). Following translation of the construct into a fusion protein, the T2A self-cleaves the fusion protein, producing two separate proteins (RAG2 and tNGFR) at a 1:1 ratio in the cell. The use of tNGFR is particularly advantageous since it enables tracking and enrichment of edited cells and has been approved for clinical applications. Further, two additional constructs with synthetic 3′-UTRs following the tNGFR were constructed, one with WPRE-BGHpA sequences and one with only the BGHpA sequence, each with a 400×800 bp homology arm pattern (CDSR_Corr_WPRE-BGHpA and CDSR_Corr_BGHpA, respectively, FIG. 3A).

While the highest rate of HDR for the CDSR-KO constructs (Example 2) was observed with an RHA of 1,600 bp (FIGS. 1B-C), correction constructs (CDSR-KI) with a 400×1,600 bp homology arm pattern could not be designed due to the limited carrying capacity (˜4.8 kb) of rAAV6 vectors. Thus, the correction constructs were designed with a 800 bp RHA.

For comparative purposes, the previously published CSI-KI construct which contained dcoRAG2 cDNA followed by the RAG2 endogenous 3′-UTR sequence along with a tNGFR reporter gene cassette under the regulation of a constitutive phosphoglycerate kinase (PGK) promoter and BGHpA sequence between 400 bp homology arms (CSI_Corr, Iancu et al., supra) was used. These four correction constructs were individually tested and highly effective locus specific HDR was observed for them all, determined by ddPCR (data not shown). For confirmation that the integration of the constructs occurred as expected, an ‘in-out’ PCR was conducted with one primer located on the tNGFR sequence and one primer downstream to the RHA (data not shown). Indeed, the observed amplified bands were consistent with the expected integration patterns and corroborated effective HDR (data not shown).

While the correction strategy relies on integrating the rAAV6 vector into the Cas9-induced break site via the homology recombination process, it is known that AAV vectors can integrate into random sites in the genome, presumed to be spontaneous DSBs, via the non-homologous DNA end joining (NHEJ) pathway. Additionally, the DSBs introduced by CRISPR-Cas9 at on- and off-target sites can also incorporate vector sequences in full or only partially, by NHEJ. In order to assess the specificity of the integration of the corrective constructs, ITR-seq, a highly effective method to detect integration of the rAAV6 vectors inverted terminal repeats (ITRs) across the genome (Breton et al, ITR-Seq, a next-generation sequencing assay, identifies genome-wide DNA editing sites in vivo following adeno-associated viral vector-mediated genome editing, BMC Genomics. 21(1):239) was used. The CSI_Corr and CDSR_Corr_Endo3′UTR constructs were tested independently, and it was found that while there was incorporation of the ITRs at the on-target site, there was relatively limited integration of the at other sites in the genome (a single off-target for each construct, data not shown).

Since the ITR-seq method is not quantitative and only detects sequences with ITR integration, amplification-free, long-range sequencing was also conducted via Oxford Nanopore Technologies (ONT) using Cas9-targeted sequencing (Gilpatrick et al., Targeted nanopore sequencing with Cas9-guided adapter ligation, Nat Biotechnol. 38(4):433-438). This method allows capturing the full scope of events, occurring upon CRISPR-Cas9-based genome editing combined with an rAAV6 vector, at the on-target locus across the cell population, without amplification bias. In particular, to quantitatively assess the extent of HDR-mediated correction versus NHEJ-based vector integration at the on-target site, Cas9-RNP digestion was used to enrich for the on-target locus, and the genome-editing products were analyzed. It was found that across three replicates, the HDR frequencies determined by ONT amplification-free sequencing and the HDR frequencies determined by ddPCR were comparable (data not shown). Additionally, NHEJ-based insertions to the cut site and partial NHEJ were kept below 5% and 9%, respectively for the CDSR_Corr_Endo3′UTR construct, and below 8% and 4%, respectively for the CSI_Corr construct, levels that are broadly comparable to prior reports.

Lastly, premature cessation of HDR when editing with the CSI_Corr construct (4.2%) was detected, due to the presence of the 3′ UTR sequence in the construct. In these cases, the non-diverged 3′ UTR sequence in the CSI_Corr constructs acts as a 3′ homology arm with the identical endogenous 3′ UTR sequence and leads to incomplete HDR.

### KI-KO Multiplex HDR Strategy for Gene Replacement in CD34⁺ HSPCs

A KI-KO strategy was utilized to engineer genotypes via multiplex HDR in HD-derived CD34⁺ HSPCs to simulate the therapeutic outcome of RAG2-SCID single-allelic correction following a gene-editing-based treatment. This strategy has two main advantages over other editing methodologies: 1) In contrast to the use of induced pluripotent stem cells (iPSCs), HD-derived CD34⁺ HSPCs are biologically authentic since they are the same cells used in hematopoietic stem cell transplantation (HSCT); and 2) Lengthy culturing protocols are insufficient since CD34⁺ HSPCs lose their regenerative ability as well as their engraftment potential after elongated culturing. Thus, the KI-KO strategy was applied in HD-derived CD34⁺ HSPCs by utilizing multiplex HDR to obtain a cell population with one allele targeted with one of the four aforementioned KI correction constructs and the other allele with a KO construct (CDSR_GFP-BGHpA_400×800 (FIG. 1) was paired with the three CDSR-KI constructs (FIG. 3), and CSI_GFP-BGHpA_400×400 (FIG. 1) was paired with the CSI_Corr construct of Iancu et al.). The RAG2-SCID disease model reported in Iancu et al. was used in order to compare the correction simulation results.

For the CSI_Corr construct, enrichment of KI-KO CD34⁺ HSPCs was achieved by sorting for biallelic double-positive tNGFR⁺/GFP⁺ expression two days post-electroporation (herein day 0) and immediately seeding the cells into the in vitro T cell differentiation (IVTD) system. However, with CDSR correction constructs, since tNGFR expression is under the regulation of the endogenous RAG2 promoter (and the RAG2 expression window occurs later in the T cell developmental process), there is no expression of tNGFR on day 0. Therefore, a novel enrichment strategy was required to isolate KI-KO cells. On day 0, the CDSR constructs were sorted only for KO GFP expression, and the GFP⁺ cells were immediately seeded into the IVTD system. On day 14 of IVTD, when RAG2 is highly expressed (thus, tNGFR is also expressed) tNGFR⁺ cells were sorted for and seeded back into the IVTD system (FIG. 3B). Additionally, all samples were sorted for CD7 expression to enrich for cells that have begun to differentiate, namely, only cells that were CD7⁺ were subjected to days 14-28 of IVTD. ddPCR was performed on genomic DNA from the KI-KO populations to confirm that the two-step enrichment method indeed led to the enrichment of a cell population with ˜100% edited alleles. Indeed, in all four correction construct multiplex HDR combinations, ˜100% of targeted alleles were found to be positive (FIG. 3C).

Additionally, since the CDSR constructs produce a fusion protein separated by a self-cleaving T2A sequence resulting in a 1:1 ratio between transgenic RAG2 and tNGFR, tNGFR MFI measurement was used as a proxy measurement for transgenic dcoRAG2 expression levels. As expected, it was observed that the MFI for CDSR_Corr_WPRE-BGHpA was 2× that of CDSR_Corr_Endo3′UTR and 1.4× greater than CDSR_Corr_BGHpA on day 28 of IVTD with a similar trend on day 14 (FIG. 3D) indicating higher expression of the dcoRAG2 in the presence of the 3′ UTR elements.

### Example 4: KI-KO HSPCs Produce CD3⁺TCRγδ⁺ and CD3⁺TCRαβ⁺ T Cells in the In Vitro T Cell Differentiation (IVTD) System

With a robust method to isolate cells with the KI-KO genotype, a validation that the expression of dcoRAG2 enabled the KI-KO cells to differentiate into CD3⁺ T cells with diverse TCR repertoires was needed, to present a proof-of-concept for gene correction. Quantitative real-time PCR (qRT-PCR) using transcript-specific primer pairs revealed that the expression of endogenous RAG2 was practically eliminated in the KI-KO populations (FIG. 4A) while robust dcoRAG2 cDNA expression was found exclusively in the KI-KO engineered cells (FIG. 4B). Additionally, when the total RAG2 mRNA levels were compared between all groups (using the RAG2-comp primers and probe), it was found that expression of the dcoRAG2 transgenes does not exceed that of the mock samples indicating that the transcription is still tightly controlled, and that the gene is not being overexpressed (FIG. 4C).

Importantly, the expression of the dcoRAG2 cDNA indeed facilitated T cell development highlighted by the successful differentiation of RAG2 KI-KO cells into CD7⁺, CD5⁺, and CD1a⁺ pre-T cells on day 14 (not shown) and CD3⁺ T cells by day 28 (FIG. 4D). Additionally, robust TCRγδ expression in the CD3⁺ population was observed by flow cytometry on day 28 with the CD3⁺TCRγδ cells presumed to be CD3⁺TCRαβ⁺ T cells (FIG. 4E). Lastly, PCR amplification using primers flanking the V-J regions of the TRG locus highlighted the successful recombination of KI-KO cells comparable to that of the mock cells on day 28 (not shown).

### Example 5: Expression of dcoRAG2 cDNA Induces Normal TCR Repertoire

Deep-sequencing analysis of TRB and TRG recombination on day 28 revealed diverse V(D)J rearrangement repertoires in the RAG2 KI-KO populations following expression of the dcoRAG2 cDNA (not shown) with no significant differences in either TRB or TRG clonotypes between the mock and RAG2 KI-KO populations as calculated by Shannon's H and Simpson's 1-D diversity indices (FIG. 5A-B). Lastly, the complementarity determining region 3 (CDR3) lengths frequency distribution was comparable in all RAG2 KI-KO and mock populations for both the TRB and TRG sequencing (not shown). CDR3 is the region of the TCR responsible for recognizing processed antigen peptides and its sequence and length varies from one clone to another. Thus, sequencing the CDR3 regions of a cell population is used as a measurement of TCR diversity. Together, these data indicate that KI and expression of the dcoRAG2 cDNA promotes successful V(D)J recombination, subsequent differentiation into CD3⁺TCRαβ⁺ and CD3⁺TCRγδ⁺ T cells, and the development of highly diverse TRB and TRG repertoires.

1-55. (canceled)

56. A system for editing of an endogenous RAG1 or RAG2 gene by replacing a gene portion sequence thereof comprised in a single exon with a transgene sequence, the system comprising:
at least one genome editing reagent designed for generating a double strand break (DSB) in a region spanning the gene portion sequence; and
a replacement nucleic acid molecule comprising a coding sequence replacement (CDSR) construct designed for serving as a template for homology-directed repair (HDR) triggered by the DSB, the CDSR construct comprising:
a. a left homology arm (LHA) comprising a sequence essentially identical to a sequence directly upstream of the gene portion sequence;
b. a right homology arm (RHA) comprising a sequence essentially identical to a sequence directly downstream of the gene portion sequence; and
c. the transgene sequence comprising an edited sequence which comprises a coding sequence (CDS) having at least 80% identity to a wild-type RAG1 or RAG2 genomic sequence corresponding to the gene portion sequence and positioned between the LHA and the RHA,
wherein following replacing the gene portion sequence, an edited gene is generated, encoding an edited gene product.

57. The system of claim 56, wherein the gene portion sequence comprises at least 50% of the CDS of the endogenous gene.

58. The system of claim 56, wherein the genome editing system is a clustered regularly interspaced short palindromic repeats (CRISPR/Cas9) system.

59. The system of claim 56, wherein the DSB is located within the gene portion sequence.

60. The system of claim 56, wherein the distance between the LHA and the DSB is longer than the distance between the RHA and the DSB and the LHA is longer than the RHA, or the distance between the RHA and the DSB is longer than the distance between the LHA and the DSB and the RHA is longer than the LHA.

61. The system of claim 56, wherein the distance between the LHA and the DSB or the distance between the RHA and the DSB is less than about 10 bp.

62. The system of claim 56, wherein the CDS of the edited sequence is not identical to a CDS of the wild-type genomic sequence, but encodes an amino acid sequence identical to the wild-type amino acid sequence.

63. The system of claim 56, wherein the transgene further comprises at least part of the 3′-UTR of the endogenous gene.

64. The system of claim 56, wherein the transgene further comprises at least one element downstream of the CDS of the edited sequence, which is not present in the endogenous gene.

65. The system of claim 56, wherein the transgene further comprises a sequence comprising a knock-in (KI) reporter gene sequence encoding a KI reporter gene product.

66. The system of claim 65, wherein the KI reporter gene product comprises a truncated cell-surface gene marker.

67. The system of claim 65, wherein the transgene is designed such that the edited sequence and the KI reporter gene sequence form a single CDS which encodes the edited gene product including the reporter gene product, and the KI reporter gene product comprises a cleavable site designed to release the KI reporter gene product from the edited gene product.

68. The system of claim 56, wherein the replacement nucleic acid molecule further comprises a recombinant adeno-associated virus serotype 6 (rAAV6).

69. The system of claim 65, further comprising a knock-out (KO) nucleic acid molecule comprising a KO construct which comprises a KO reporter gene sequence encoding a KO reporter gene product, wherein the KO construct is designed for incorporating the KO reporter gene sequence into an integration region in the endogenous gene sequence, thereby preventing expression of an endogenous gene product, and the KO reporter gene is different from the KI reporter gene.

70. An ex vivo or in vitro method for editing of an endogenous RAG1 or RAG2 gene by replacing a gene portion sequence thereof comprised in a single exon with a transgene sequence, the method comprising:
a. providing stem cells or progenitor cells suitable for gene targeting;
b. contacting the cells with at least one genome editing reagent thereby generating a double strand break (DSB) in a region spanning the gene portion sequence;
c. contacting the cells with a replacement nucleic acid molecule comprising a coding sequence replacement (CDSR) construct serving as a template for homology-directed repair (HDR) triggered by the DSB, the CDSR construct comprising:
i. a left homology arm (LHA) comprising a sequence essentially identical to a sequence directly upstream of the gene portion sequence,
ii. a right homology arm (RHA) comprising a sequence essentially identical to a sequence directly downstream of the gene portion sequence, and
iii. the transgene sequence comprising an edited sequence which comprises a coding sequence (CDS) having at least 80% identity to a wild-type genomic sequence corresponding to the RAG1 or RAG2 gene portion sequence and positioned between the LHA and the RHA; and
d. isolating edited cells comprising an edited gene encoding an edited gene product.

71. The method of claim 70, wherein the transgene further comprises a sequence comprising a knock-in (KI) reporter gene sequence encoding a KI reporter gene product and isolating the edited cells in step (d) comprises enriching for cells expressing the KI reporter gene product.

72. The method of claim 71, wherein the transgene is designed such that the edited sequence and the KI reporter gene sequence form a single coding sequence (CDS) which encodes the edited gene product including the KI reporter gene product, and the KI reporter gene product comprises a cleavable site designed to release the KI reporter gene product from the edited gene product.

73. The method of claim 70, further comprising at step (c) contacting the cells with a knock-out (KO) nucleic acid molecule comprising a KO construct which comprises a KO reporter gene encoding a KO reporter gene product, wherein the KO construct is designed for incorporating the KO reporter gene into an integration region in the endogenous gene sequence, thereby preventing expression of an endogenous gene product, and the KO reporter gene is different from the KI reporter gene.

74. The method of claim 73, wherein isolating the cells in step (d) comprises enriching for cells expressing both the KI reporter gene product and the KO reporting gene product.

75. A method of treating a disease or a disorder caused by a mutation in an endogenous gene in a subject in need thereof, comprising administering to the subject the edited cells comprising the edited gene obtained by the method claim 70.


SEQUENCE LISTING (84 sequences)

SEQ ID NO: 1 - RNA, 20 nt, synthetic construct
  source 1..20: mol_type = other RNA
tgagaagcctggctgaatta

SEQ ID NO: 2 - RNA, 20 nt, synthetic construct
  source 1..20: mol_type = other RNA
ttgactcagggttccaccca

SEQ ID NO: 3 - DNA, 492 bp, synthetic construct
  source 1..492: mol_type = other DNA
ccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 4 - DNA, 681 bp, synthetic construct
  source 1..681: mol_type = other DNA
  CDS 1..681: protein_id = 68; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctag

SEQ ID NO: 5 - DNA, 227 bp, synthetic construct
  source 1..227: mol_type = other DNA
agcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggct

SEQ ID NO: 6 - DNA, 592 bp, synthetic construct
  source 1..592: mol_type = other DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 7 - DNA, 521 bp, synthetic construct
  source 1..521: mol_type = other DNA
gaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 8 - DNA, 843 bp, synthetic construct
  source 1..843: mol_type = other DNA
  CDS 1..843: protein_id = 69; translation = MGAGATGRAMDGPRLLLLLLLGVSLGGAKEACPTGLYTHSGECC KACNLGEGVAQPCGANQTVCEPCLDSVTFSDVVSATEPCKPCTECVGLQSMSAPCVEA DDAVCRCAYGYYQDETTGRCEACRVCEAGSGLVFSCQDKQNTVCEECPDGTYSDEANH VDPCLPCTVCEDTERQLRECTRWADAECEEIPGRWITRSTPPEGSDSTAPSTQEPEAP PEQDLIASTVAGVVTTVMGSSQPVVTRGTTDNLIPVYCSILAAVVVGLVAYIAFKRWN RGIL
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctag

SEQ ID NO: 9 - DNA, 63 bp, synthetic construct
  source 1..63: mol_type = other DNA
ggctccggcgagggcagaggaagtcttctaacatgcggtgacgtggaggagaatcccggccca

SEQ ID NO: 10 - DNA, 400 bp, synthetic construct
  source 1..400: mol_type = other DNA
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaa

SEQ ID NO: 11 - DNA, 400 bp, synthetic construct
  source 1..400: mol_type = other DNA
ttcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccg

SEQ ID NO: 12 - DNA, 400 bp, synthetic construct
  source 1..400: mol_type = other DNA
ttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaaga

SEQ ID NO: 13 - DNA, 800 bp, synthetic construct
  source 1..800: mol_type = other DNA
ttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactct

SEQ ID NO: 14 - DNA, 1600 bp, synthetic construct
  source 1..1600: mol_type = other DNA
ttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactctggaaaaaggaagtgagtaagaatacaataagaaaggaccaccaaataaatctcatggagaagaataatattatttcccaaaataatgtaagtttaaagaatttcatgacatttacaggcagaagatttaaatggaggaaaagaaatgtatttgtgtgagtgtgtgtgtgtgtgtgtgtgtgtgtgtagttaacatgtggagattgctgccagtgaatattataaaatcagtgatcttgccaaggtctaattagacactcgctaggatgaaactagttaaaatgactgttaattttaagggttcaagacatcagttgataactagatgaccttagaaacaaatgtctttcctcctgaaatattttccggaaaaaaaaattttctggaaaaaccttatcttaagagcttcagccacagttacagtgaagactcttactcctcactgaaagtctacagtgtgtaaggtacaactaacaaatttacgggaaacatgaattatgcaagagatgaaacgctggagtacaagttcttctgctgaaagttccatgtccccagatactgaaattaactttgataaactgaatcatattcactctgtttcaataatgttgctcgctgaatattcctgttgaacaattgttgcacattgatttgatcttttgttgtttccatcacctatataaaaataataaaatataaaaatcttctatttatattgatttactctaattattaagatatgttacctaaataacggcaaaattaaactaattttttcaggatcactccactttgattttt

SEQ ID NO: 15 - DNA, 801 bp, synthetic construct
  source 1..801: mol_type = other DNA
tgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacc

SEQ ID NO: 16 - DNA, 801 bp, synthetic construct
  source 1..801: mol_type = other DNA
caccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatct

SEQ ID NO: 17 - DNA, 800 bp, synthetic construct
  source 1..800: mol_type = other DNA
gtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgat

SEQ ID NO: 18 - DNA, 1600 bp, synthetic construct
  source 1..1600: mol_type = other DNA
gtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgc

SEQ ID NO: 19 - DNA, 2000 bp, synthetic construct
  source 1..2000: mol_type = other DNA
gtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatc

SEQ ID NO: 20 - DNA, 1538 bp, synthetic construct
  source 1..1538: mol_type = other DNA
  CDS 3..1538: protein_id = 67; translation = QPGFSLMNFDGQVFFFGQKGWPKRSCPTGVFHLDVKHNHVKLKP TIFSKDSCYLPPLRYPATCTFKGSLESEKHQYIIHGGKTPNNEVSDKIYVMSIVCKNN KKVTFRCTEKDLVGDVPEARYGHSINVVYSRGKSMGVLFGGRSYMPSTHRTTEKWNSV ADCLPCVFLVDFEFGCATSYILPELQDGLSFHVSIAKNDTIYILGGHSLANNIRPANL YRIRVDLPLGSPAVNCTVLPGGISVSSAILTQTNNDEFVIVGGYQLENQKRMICNIIS LEDNKIEIREMETPDWTPDIKHSKIWFGSNMGNGTVFLGIPGDNKQVVSEGFYFYMLK CAEDDTNEEQTTFTNSQTSTEDPGDSTPFEDSEEFCFSAEANSFDGDDEFDTYNEDDE EDESETGYWITCCPTCDVDINTWVPFYSTELNKPAMIYCSHGDGHWVHAQCMDLAERT LIHLSAGSNKYYCNEHVEIARALHTPQRVLPLKKPPMKSLRKKGSGKILTPAKKSFLR RLFD
tccagcccggcttcagcctcatgaacttcgacggccaggtgttttttttcggccagaagggatggcctaagaggagctgtcctaccggcgtgttccacctcgacgtgaagcacaatcacgtgaagctcaaacccaccatctttagcaaagacagctgttatctgccccccctgagatatcccgctacctgtacctttaagggatccctggaaagcgagaaacaccagtatattattcacggcggcaagacccccaataacgaagtgagcgacaaaatctacgtgatgagcatcgtgtgtaaaaataataagaaagtgaccttcagatgtaccgaaaaggatctggtgggcgacgtgcccgaggctaggtacggccacagcatcaacgtcgtctattccagaggcaagagcatgggcgtgctgttcggcggcagaagctatatgcccagcacacataggacaaccgagaagtggaacagcgtggccgattgtctcccttgcgtgtttctcgtcgacttcgagttcggctgcgccaccagctatatcctgcccgagctgcaagacggcctgagcttccacgtgagcatcgctaagaacgatacaatttacatcctgggcggccacagcctggctaacaacattagacccgctaatctctataggatcagagtggacctgcctctcggctcccccgccgtcaactgtaccgtgctgcccggcggcattagcgtgagcagcgccattctcacccagaccaataacgacgagttcgtgatcgtgggcggataccaactggagaaccagaagaggatgatttgtaatattattagcctggaagataataaaatcgagatcagagaaatggaaacacccgactggacacccgatatcaaacattccaaaatctggttcggctccaatatgggcaacggcaccgtgttcctgggaatccccggcgataacaagcaggtggtgagcgagggcttttacttttacatgctgaagtgcgccgaggacgacaccaacgaggaacaaaccacctttaccaatagccagaccagcaccgaggaccccggcgacagcacccctttcgaggatagcgaggagttctgctttagcgccgaggccaacagcttcgacggcgacgacgagttcgatacatacaacgaggacgacgaggaggacgaaagcgaaaccggatattggatcacctgttgtcccacctgcgacgtcgacattaatacctgggtgcccttttacagcaccgaactgaataagcctgctatgatttattgtagccacggcgacggccattgggtgcacgcccaatgtatggacctcgccgagagaaccctgattcacctcagcgccggctccaataaatactattgtaacgaacacgtcgaaatcgccagggccctgcatacccctcagagggtgctgcctctgaagaaaccccccatgaagagcctgagaaagaagggcagcggcaagattctgacccccgctaaaaagagcttcctgaggagactgttcgac

SEQ ID NO: 21 - DNA, 640 bp, synthetic construct
  source 1..640: mol_type = other DNA
ttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatc

SEQ ID NO: 22 - DNA, 2311 bp, synthetic construct
  source 1..2311: mol_type = other DNA
  CDS 946..1626: protein_id = 70; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccttcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccg

SEQ ID NO: 23 - DNA, 2311 bp, synthetic construct
  source 1..2311: mol_type = other DNA
  CDS 946..1626: protein_id = 71; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaaga

SEQ ID NO: 24 - DNA, 2711 bp, synthetic construct
  source 1..2711: mol_type = other DNA
  CDS 946..1626: protein_id = 72; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactct

SEQ ID NO: 25 - DNA, 3511 bp, synthetic construct
  source 1..3511: mol_type = other DNA
  CDS 946..1626: protein_id = 73; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactctggaaaaaggaagtgagtaagaatacaataagaaaggaccaccaaataaatctcatggagaagaataatattatttcccaaaataatgtaagtttaaagaatttcatgacatttacaggcagaagatttaaatggaggaaaagaaatgtatttgtgtgagtgtgtgtgtgtgtgtgtgtgtgtgtgtagttaacatgtggagattgctgccagtgaatattataaaatcagtgatcttgccaaggtctaattagacactcgctaggatgaaactagttaaaatgactgttaattttaagggttcaagacatcagttgataactagatgaccttagaaacaaatgtctttcctcctgaaatattttccggaaaaaaaaattttctggaaaaaccttatcttaagagcttcagccacagttacagtgaagactcttactcctcactgaaagtctacagtgtgtaaggtacaactaacaaatttacgggaaacatgaattatgcaagagatgaaacgctggagtacaagttcttctgctgaaagttccatgtccccagatactgaaattaactttgataaactgaatcatattcactctgtttcaataatgttgctcgctgaatattcctgttgaacaattgttgcacattgatttgatcttttgttgtttccatcacctatataaaaataataaaatataaaaatcttctatttatattgatttactctaattattaagatatgttacctaaataacggcaaaattaaactaattttttcaggatcactccactttgattttt

SEQ ID NO: 26 - DNA, 3226 bp, synthetic construct
  source 1..3226: mol_type = other DNA
  CDS 946..1626: protein_id = 74; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactctggaaaaaggaagtgagtaagaatacaataagaaaggaccaccaaataaatctcatggagaagaataatattatttcccaaaataatgtaagtttaaagaatttcatgacatttacaggcagaagatttaaatggaggaaaagaaatgtatttgtgtgagtgtgtgtgtgtgtgtgtgtgtgtgtgtagttaacatgtggagattgctgccagtgaatattataaaatcagtgatcttgccaaggtctaattagacactcgctaggatgaaactagttaaaatgactgttaattttaagggttcaagacatcagttgataactagatgaccttagaaacaaatgtctttcctcctgaaatattttccggaaaaaaaaattttctggaaaaaccttatcttaagagcttcagccacagttacagtgaagactcttactcctcactgaaagtctacagtgtgtaaggtacaactaacaaatttacgggaaacatgaattatgcaagagatgaaacgctggagtacaagttcttctgctgaaagttccatgtccccagatactgaaattaactttgataaactgaatcatattcactctgtttcaataatgttgctcgctgaatattcctgttgaacaattgttgcacattgatttgatcttttgttgtttccatcacctatataaaaataataaaatataaaaatcttctatttatattgatttactctaattattaagatatgttacctaaataacggcaaaattaaactaattttttcaggatcactccactttgattttt

SEQ ID NO: 27 - DNA, 4105 bp, synthetic construct
  source 1..4105: mol_type = other DNA
  CDS 946..1626: protein_id = 75; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaaattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagggcgcgcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgccttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactctggaaaaaggaagtgagtaagaatacaataagaaaggaccaccaaataaatctcatggagaagaataatattatttcccaaaataatgtaagtttaaagaatttcatgacatttacaggcagaagatttaaatggaggaaaagaaatgtatttgtgtgagtgtgtgtgtgtgtgtgtgtgtgtgtgtagttaacatgtggagattgctgccagtgaatattataaaatcagtgatcttgccaaggtctaattagacactcgctaggatgaaactagttaaaatgactgttaattttaagggttcaagacatcagttgataactagatgaccttagaaacaaatgtctttcctcctgaaatattttccggaaaaaaaaattttctggaaaaaccttatcttaagagcttcagccacagttacagtgaagactcttactcctcactgaaagtctacagtgtgtaaggtacaactaacaaatttacgggaaacatgaattatgcaagagatgaaacgctggagtacaagttcttctgctgaaagttccatgtccccagatactgaaattaactttgataaactgaatcatattcactctgtttcaataatgttgctcgctgaatattcctgttgaacaattgttgcacattgatttgatcttttgttgtttccatcacctatataaaaataataaaatataaaaatcttctatttatattgatttactctaattattaagatatgttacctaaataacggcaaaattaaactaattttttcaggatcactccactttgattttt

SEQ ID NO: 28 - DNA, 4572 bp, synthetic construct
  source 1..4572: mol_type = other DNA
  CDS 358..1941: protein_id = 80; translation = MSLQMVTVSNNIALIQPGFSLMNFDGQVFFFGQKGWPKRSCPTG VFHLDVKHNHVKLKPTIFSKDSCYLPPLRYPATCTFKGSLESEKHQYIIHGGKTPNNE VSDKIYVMSIVCKNNKKVTFRCTEKDLVGDVPEARYGHSINVVYSRGKSMGVLFGGRS YMPSTHRTTEKWNSVADCLPCVFLVDFEFGCATSYILPELQDGLSFHVSIAKNDTIYI LGGHSLANNIRPANLYRIRVDLPLGSPAVNCTVLPGGISVSSAILTQTNNDEFVIVGG YQLENQKRMICNIISLEDNKIEIREMETPDWTPDIKHSKIWFGSNMGNGTVFLGIPGD NKQVVSEGFYFYMLKCAEDDTNEEQTTFTNSQTSTEDPGDSTPFEDSEEFCFSAEANS FDGDDEFDTYNEDDEEDESETGYWITCCPTCDVDINTWVPFYSTELNKPAMIYCSHGD GHWVHAQCMDLAERTLIHLSAGSNKYYCNEHVEIARALHTPQRVLPLKKPPMKSLRKK GSGKILTPAKKSFLRRLFD
  CDS 3103..3945: protein_id = 81; translation = MGAGATGRAMDGPRLLLLLLLGVSLGGAKEACPTGLYTHSGECC KACNLGEGVAQPCGANQTVCEPCLDSVTFSDVVSATEPCKPCTECVGLQSMSAPCVEA DDAVCRCAYGYYQDETTGRCEACRVCEAGSGLVFSCQDKQNTVCEECPDGTYSDEANH VDPCLPCTVCEDTERQLRECTRWADAECEEIPGRWITRSTPPEGSDSTAPSTQEPEAP PEQDLIASTVAGVVTTVMGSSQPVVTRGTTDNLIPVYCSILAAVVVGLVAYIAFKRWN RGIL
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaatccagcccggcttcagcctcatgaacttcgacggccaggtgttttttttcggccagaagggatggcctaagaggagctgtcctaccggcgtgttccacctcgacgtgaagcacaatcacgtgaagctcaaacccaccatctttagcaaagacagctgttatctgccccccctgagatatcccgctacctgtacctttaagggatccctggaaagcgagaaacaccagtatattattcacggcggcaagacccccaataacgaagtgagcgacaaaatctacgtgatgagcatcgtgtgtaaaaataataagaaagtgaccttcagatgtaccgaaaaggatctggtgggcgacgtgcccgaggctaggtacggccacagcatcaacgtcgtctattccagaggcaagagcatgggcgtgctgttcggcggcagaagctatatgcccagcacacataggacaaccgagaagtggaacagcgtggccgattgtctcccttgcgtgtttctcgtcgacttcgagttcggctgcgccaccagctatatcctgcccgagctgcaagacggcctgagcttccacgtgagcatcgctaagaacgatacaatttacatcctgggcggccacagcctggctaacaacattagacccgctaatctctataggatcagagtggacctgcctctcggctcccccgccgtcaactgtaccgtgctgcccggcggcattagcgtgagcagcgccattctcacccagaccaataacgacgagttcgtgatcgtgggcggataccaactggagaaccagaagaggatgatttgtaatattattagcctggaagataataaaatcgagatcagagaaatggaaacacccgactggacacccgatatcaaacattccaaaatctggttcggctccaatatgggcaacggcaccgtgttcctgggaatccccggcgataacaagcaggtggtgagcgagggcttttacttttacatgctgaagtgcgccgaggacgacaccaacgaggaacaaaccacctttaccaatagccagaccagcaccgaggaccccggcgacagcacccctttcgaggatagcgaggagttctgctttagcgccgaggccaacagcttcgacggcgacgacgagttcgatacatacaacgaggacgacgaggaggacgaaagcgaaaccggatattggatcacctgttgtcccacctgcgacgtcgacattaatacctgggtgcccttttacagcaccgaactgaataagcctgctatgatttattgtagccacggcgacggccattgggtgcacgcccaatgtatggacctcgccgagagaaccctgattcacctcagcgccggctccaataaatactattgtaacgaacacgtcgaaatcgccagggccctgcatacccctcagagggtgctgcctctgaagaaaccccccatgaagagcctgagaaagaagggcagcggcaagattctgacccccgctaaaaagagcttcctgaggagactgttcgactgattttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctagagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctttcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccg

SEQ ID NO: 29 - DNA, 3644 bp, synthetic construct
  source 1..3644: mol_type = other DNA
  CDS 358..2844: protein_id = 82; translation = MSLQMVTVSNNIALIQPGFSLMNFDGQVFFFGQKGWPKRSCPTG VFHLDVKHNHVKLKPTIFSKDSCYLPPLRYPATCTFKGSLESEKHQYIIHGGKTPNNE VSDKIYVMSIVCKNNKKVTFRCTEKDLVGDVPEARYGHSINVVYSRGKSMGVLFGGRS YMPSTHRTTEKWNSVADCLPCVFLVDFEFGCATSYILPELQDGLSFHVSIAKNDTIYI LGGHSLANNIRPANLYRIRVDLPLGSPAVNCTVLPGGISVSSAILTQTNNDEFVIVGG YQLENQKRMICNIISLEDNKIEIREMETPDWTPDIKHSKIWFGSNMGNGTVFLGIPGD NKQVVSEGFYFYMLKCAEDDTNEEQTTFTNSQTSTEDPGDSTPFEDSEEFCFSAEANS FDGDDEFDTYNEDDEEDESETGYWITCCPTCDVDINTWVPFYSTELNKPAMIYCSHGD GHWVHAQCMDLAERTLIHLSAGSNKYYCNEHVEIARALHTPQRVLPLKKPPMKSLRKK GSGKILTPAKKSFLRRLFDGSGEGRGSLLTCGDVEENPGPMGAGATGRAMDGPRLLLL LLLGVSLGGAKEACPTGLYTHSGECCKACNLGEGVAQPCGANQTVCEPCLDSVTFSDV VSATEPCKPCTECVGLQSMSAPCVEADDAVCRCAYGYYQDETTGRCEACRVCEAGSGL VFSCQDKQNTVCEECPDGTYSDEANHVDPCLPCTVCEDTERQLRECTRWADAECEEIP GRWITRSTPPEGSDSTAPSTQEPEAPPEQDLIASTVAGVVTTVMGSSQPVVTRGTTDN LIPVYCSILAAVVVGLVAYIAFKRWNRGIL
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaatccagcccggcttcagcctcatgaacttcgacggccaggtgttttttttcggccagaagggatggcctaagaggagctgtcctaccggcgtgttccacctcgacgtgaagcacaatcacgtgaagctcaaacccaccatctttagcaaagacagctgttatctgccccccctgagatatcccgctacctgtacctttaagggatccctggaaagcgagaaacaccagtatattattcacggcggcaagacccccaataacgaagtgagcgacaaaatctacgtgatgagcatcgtgtgtaaaaataataagaaagtgaccttcagatgtaccgaaaaggatctggtgggcgacgtgcccgaggctaggtacggccacagcatcaacgtcgtctattccagaggcaagagcatgggcgtgctgttcggcggcagaagctatatgcccagcacacataggacaaccgagaagtggaacagcgtggccgattgtctcccttgcgtgtttctcgtcgacttcgagttcggctgcgccaccagctatatcctgcccgagctgcaagacggcctgagcttccacgtgagcatcgctaagaacgatacaatttacatcctgggcggccacagcctggctaacaacattagacccgctaatctctataggatcagagtggacctgcctctcggctcccccgccgtcaactgtaccgtgctgcccggcggcattagcgtgagcagcgccattctcacccagaccaataacgacgagttcgtgatcgtgggcggataccaactggagaaccagaagaggatgatttgtaatattattagcctggaagataataaaatcgagatcagagaaatggaaacacccgactggacacccgatatcaaacattccaaaatctggttcggctccaatatgggcaacggcaccgtgttcctgggaatccccggcgataacaagcaggtggtgagcgagggcttttacttttacatgctgaagtgcgccgaggacgacaccaacgaggaacaaaccacctttaccaatagccagaccagcaccgaggaccccggcgacagcacccctttcgaggatagcgaggagttctgctttagcgccgaggccaacagcttcgacggcgacgacgagttcgatacatacaacgaggacgacgaggaggacgaaagcgaaaccggatattggatcacctgttgtcccacctgcgacgtcgacattaatacctgggtgcccttttacagcaccgaactgaataagcctgctatgatttattgtagccacggcgacggccattgggtgcacgcccaatgtatggacctcgccgagagaaccctgattcacctcagcgccggctccaataaatactattgtaacgaacacgtcgaaatcgccagggccctgcatacccctcagagggtgctgcctctgaagaaaccccccatgaagagcctgagaaagaagggcagcggcaagattctgacccccgctaaaaagagcttcctgaggagactgttcgacggctccggcgagggcagaggaagtcttctaacatgcggtgacgtggaggagaatcccggcccaatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactct

SEQ ID NO: 30 - DNA, 3894 bp, synthetic construct
  source 1..3894: mol_type = other DNA
  CDS 358..2844: protein_id = 83; translation = MSLQMVTVSNNIALIQPGFSLMNFDGQVFFFGQKGWPKRSCPTG VFHLDVKHNHVKLKPTIFSKDSCYLPPLRYPATCTFKGSLESEKHQYIIHGGKTPNNE VSDKIYVMSIVCKNNKKVTFRCTEKDLVGDVPEARYGHSINVVYSRGKSMGVLFGGRS YMPSTHRTTEKWNSVADCLPCVFLVDFEFGCATSYILPELQDGLSFHVSIAKNDTIYI LGGHSLANNIRPANLYRIRVDLPLGSPAVNCTVLPGGISVSSAILTQTNNDEFVIVGG YQLENQKRMICNIISLEDNKIEIREMETPDWTPDIKHSKIWFGSNMGNGTVFLGIPGD NKQVVSEGFYFYMLKCAEDDTNEEQTTFTNSQTSTEDPGDSTPFEDSEEFCFSAEANS FDGDDEFDTYNEDDEEDESETGYWITCCPTCDVDINTWVPFYSTELNKPAMIYCSHGD GHWVHAQCMDLAERTLIHLSAGSNKYYCNEHVEIARALHTPQRVLPLKKPPMKSLRKK GSGKILTPAKKSFLRRLFDGSGEGRGSLLTCGDVEENPGPMGAGATGRAMDGPRLLLL LLLGVSLGGAKEACPTGLYTHSGECCKACNLGEGVAQPCGANQTVCEPCLDSVTFSDV VSATEPCKPCTECVGLQSMSAPCVEADDAVCRCAYGYYQDETTGRCEACRVCEAGSGL VFSCQDKQNTVCEECPDGTYSDEANHVDPCLPCTVCEDTERQLRECTRWADAECEEIP GRWITRSTPPEGSDSTAPSTQEPEAPPEQDLIASTVAGVVTTVMGSSQPVVTRGTTDN LIPVYCSILAAVVVGLVAYIAFKRWNRGIL
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaatccagcccggcttcagcctcatgaacttcgacggccaggtgttttttttcggccagaagggatggcctaagaggagctgtcctaccggcgtgttccacctcgacgtgaagcacaatcacgtgaagctcaaacccaccatctttagcaaagacagctgttatctgccccccctgagatatcccgctacctgtacctttaagggatccctggaaagcgagaaacaccagtatattattcacggcggcaagacccccaataacgaagtgagcgacaaaatctacgtgatgagcatcgtgtgtaaaaataataagaaagtgaccttcagatgtaccgaaaaggatctggtgggcgacgtgcccgaggctaggtacggccacagcatcaacgtcgtctattccagaggcaagagcatgggcgtgctgttcggcggcagaagctatatgcccagcacacataggacaaccgagaagtggaacagcgtggccgattgtctcccttgcgtgtttctcgtcgacttcgagttcggctgcgccaccagctatatcctgcccgagctgcaagacggcctgagcttccacgtgagcatcgctaagaacgatacaatttacatcctgggcggccacagcctggctaacaacattagacccgctaatctctataggatcagagtggacctgcctctcggctcccccgccgtcaactgtaccgtgctgcccggcggcattagcgtgagcagcgccattctcacccagaccaataacgacgagttcgtgatcgtgggcggataccaactggagaaccagaagaggatgatttgtaatattattagcctggaagataataaaatcgagatcagagaaatggaaacacccgactggacacccgatatcaaacattccaaaatctggttcggctccaatatgggcaacggcaccgtgttcctgggaatccccggcgataacaagcaggtggtgagcgagggcttttacttttacatgctgaagtgcgccgaggacgacaccaacgaggaacaaaccacctttaccaatagccagaccagcaccgaggaccccggcgacagcacccctttcgaggatagcgaggagttctgctttagcgccgaggccaacagcttcgacggcgacgacgagttcgatacatacaacgaggacgacgaggaggacgaaagcgaaaccggatattggatcacctgttgtcccacctgcgacgtcgacattaatacctgggtgcccttttacagcaccgaactgaataagcctgctatgatttattgtagccacggcgacggccattgggtgcacgcccaatgtatggacctcgccgagagaaccctgattcacctcagcgccggctccaataaatactattgtaacgaacacgtcgaaatcgccagggccctgcatacccctcagagggtgctgcctctgaagaaaccccccatgaagagcctgagaaagaagggcagcggcaagattctgacccccgctaaaaagagcttcctgaggagactgttcgacggctccggcgagggcagaggaagtcttctaacatgcggtgacgtggaggagaatcccggcccaatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctagctcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactct

SEQ ID NO: 31 - DNA, 4500 bp, synthetic construct
  source 1..4500: mol_type = other DNA
  CDS 358..2844: protein_id = 84; translation = MSLQMVTVSNNIALIQPGFSLMNFDGQVFFFGQKGWPKRSCPTG VFHLDVKHNHVKLKPTIFSKDSCYLPPLRYPATCTFKGSLESEKHQYIIHGGKTPNNE VSDKIYVMSIVCKNNKKVTFRCTEKDLVGDVPEARYGHSINVVYSRGKSMGVLFGGRS YMPSTHRTTEKWNSVADCLPCVFLVDFEFGCATSYILPELQDGLSFHVSIAKNDTIYI LGGHSLANNIRPANLYRIRVDLPLGSPAVNCTVLPGGISVSSAILTQTNNDEFVIVGG YQLENQKRMICNIISLEDNKIEIREMETPDWTPDIKHSKIWFGSNMGNGTVFLGIPGD NKQVVSEGFYFYMLKCAEDDTNEEQTTFTNSQTSTEDPGDSTPFEDSEEFCFSAEANS FDGDDEFDTYNEDDEEDESETGYWITCCPTCDVDINTWVPFYSTELNKPAMIYCSHGD GHWVHAQCMDLAERTLIHLSAGSNKYYCNEHVEIARALHTPQRVLPLKKPPMKSLRKK GSGKILTPAKKSFLRRLFDGSGEGRGSLLTCGDVEENPGPMGAGATGRAMDGPRLLLL LLLGVSLGGAKEACPTGLYTHSGECCKACNLGEGVAQPCGANQTVCEPCLDSVTFSDV VSATEPCKPCTECVGLQSMSAPCVEADDAVCRCAYGYYQDETTGRCEACRVCEAGSGL VFSCQDKQNTVCEECPDGTYSDEANHVDPCLPCTVCEDTERQLRECTRWADAECEEIP GRWITRSTPPEGSDSTAPSTQEPEAPPEQDLIASTVAGVVTTVMGSSQPVVTRGTTDN LIPVYCSILAAVVVGLVAYIAFKRWNRGIL
tccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaccatcagaaactatgtctctgcagatggtaacagtcagtaataacatagccttaatccagcccggcttcagcctcatgaacttcgacggccaggtgttttttttcggccagaagggatggcctaagaggagctgtcctaccggcgtgttccacctcgacgtgaagcacaatcacgtgaagctcaaacccaccatctttagcaaagacagctgttatctgccccccctgagatatcccgctacctgtacctttaagggatccctggaaagcgagaaacaccagtatattattcacggcggcaagacccccaataacgaagtgagcgacaaaatctacgtgatgagcatcgtgtgtaaaaataataagaaagtgaccttcagatgtaccgaaaaggatctggtgggcgacgtgcccgaggctaggtacggccacagcatcaacgtcgtctattccagaggcaagagcatgggcgtgctgttcggcggcagaagctatatgcccagcacacataggacaaccgagaagtggaacagcgtggccgattgtctcccttgcgtgtttctcgtcgacttcgagttcggctgcgccaccagctatatcctgcccgagctgcaagacggcctgagcttccacgtgagcatcgctaagaacgatacaatttacatcctgggcggccacagcctggctaacaacattagacccgctaatctctataggatcagagtggacctgcctctcggctcccccgccgtcaactgtaccgtgctgcccggcggcattagcgtgagcagcgccattctcacccagaccaataacgacgagttcgtgatcgtgggcggataccaactggagaaccagaagaggatgatttgtaatattattagcctggaagataataaaatcgagatcagagaaatggaaacacccgactggacacccgatatcaaacattccaaaatctggttcggctccaatatgggcaacggcaccgtgttcctgggaatccccggcgataacaagcaggtggtgagcgagggcttttacttttacatgctgaagtgcgccgaggacgacaccaacgaggaacaaaccacctttaccaatagccagaccagcaccgaggaccccggcgacagcacccctttcgaggatagcgaggagttctgctttagcgccgaggccaacagcttcgacggcgacgacgagttcgatacatacaacgaggacgacgaggaggacgaaagcgaaaccggatattggatcacctgttgtcccacctgcgacgtcgacattaatacctgggtgcccttttacagcaccgaactgaataagcctgctatgatttattgtagccacggcgacggccattgggtgcacgcccaatgtatggacctcgccgagagaaccctgattcacctcagcgccggctccaataaatactattgtaacgaacacgtcgaaatcgccagggccctgcatacccctcagagggtgctgcctctgaagaaaccccccatgaagagcctgagaaagaagggcagcggcaagattctgacccccgctaaaaagagcttcctgaggagactgttcgacggctccggcgagggcagaggaagtcttctaacatgcggtgacgtggaggagaatcccggcccaatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctagctcgagggcgcgcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctaagcttttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaagtttgacatgttttcctgaattacttgtatgcattttctttaaggatttcccctccccattatatgatgcatgaatgatgtgccaaagtcagtcatttgaactaattatttgtgagaattttcatggaaaactggagcccattgctacttagtactct

SEQ ID NO: 32 - DNA, 3067 bp, synthetic construct
  source 1..3067: mol_type = other DNA
  CDS 1331..2011: protein_id = 76; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatct

SEQ ID NO: 33 - DNA, 3066 bp, synthetic construct
  source 1..3066: mol_type = other DNA
  CDS 1331..2011: protein_id = 77; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgat

SEQ ID NO: 34 - DNA, 3866 bp, synthetic construct
  source 1..3866: mol_type = other DNA
  CDS 1331..2011: protein_id = 78; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgc

SEQ ID NO: 35 - DNA, 4266 bp, synthetic construct
  source 1..4266: mol_type = other DNA
  CDS 1331..2011: protein_id = 79; translation = MPAMKIECRITGTLNGVEFELVGGGEGTPEQGRMTNKMKSTKGA LTFSPYLLSHVMGYGFYHFGTYPSGYENPFLHAINNGGYTNTRIEKYEDGGVLHVSFS YRYEAGRVIGDFKVVGTGFPEDSVIFTDKIIRSNATVEHLHPMGDNVLVGSFARTFSL RDGGYYSFVVDSHMHFKSAIHPSILQNGGPMFAFRRVEELHSNTELGIVEYQHAFKTP IAFARSRV
tgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcaggtacctcagccagcatggcagcctctttcccacctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatc

SEQ ID NO: 36 - DNA, 21 bp, synthetic construct
  source 1..21: mol_type = other DNA
ctctcccagagcaacaaagac

SEQ ID NO: 37 - DNA, 21 bp, synthetic construct
  source 1..21: mol_type = other DNA
tttccatgccttgcaaaatgg

SEQ ID NO: 38 - DNA, 24 bp, synthetic construct
  source 1..24: mol_type = other DNA
cctacaggtggggtctttcattcc

SEQ ID NO: 39 - DNA, 18 bp, synthetic construct
  source 1..18: mol_type = other DNA
tctcccagagcaacaaag

SEQ ID NO: 40 - DNA, 19 bp, synthetic construct
  source 1..19: mol_type = other DNA
gaagttcatgaggctgaag

SEQ ID NO: 41 - DNA, 20 bp, synthetic construct
  source 1..20: mol_type = other DNA
catagccttaatccagcccg

SEQ ID NO: 42 - DNA, 21 bp, synthetic construct
  source 1..21: mol_type = other DNA
cagtgactttcaggatgacct

SEQ ID NO: 43 - DNA, 18 bp, synthetic construct
  source 1..18: mol_type = other DNA
cagctagcttgccaaacc

SEQ ID NO: 44 - DNA, 19 bp, synthetic construct
  source 1..19: mol_type = other DNA
ccatgctggctgaggtacc

SEQ ID NO: 45 - DNA, 19 bp, synthetic construct
  source 1..19: mol_type = other DNA
gctgtatgaatccaggtcc

SEQ ID NO: 46 - DNA, 19 bp, synthetic construct
  source 1..19: mol_type = other DNA
cctcctggctgagaaaaag

SEQ ID NO: 47 - DNA, 26 bp, synthetic construct
  source 1..26: mol_type = other DNA
tgtttcctccaggataaggcagctgt

SEQ ID NO: 48 - DNA, 24 bp, synthetic construct
  source 1..24: mol_type = other DNA
ccaagtgctgacaattaatacctg

SEQ ID NO: 49 - DNA, 24 bp, synthetic construct
  source 1..24: mol_type = other DNA
gacatggttatgctttacatccag

SEQ ID NO: 50 - DNA, 29 bp, synthetic construct
  source 1..29: mol_type = other DNA
catcagtgagaagcctggctgaattaagg

SEQ ID NO: 51 - DNA, 22 bp, synthetic construct
  source 1..22: mol_type = other DNA
caagtgctgacaattaatacct

SEQ ID NO: 52 - DNA, 19 bp, synthetic construct
  source 1..19: mol_type = other DNA
gaagttcatgaggctgaag

SEQ ID NO: 53 - DNA, 20 bp, synthetic construct
  source 1..20: mol_type = other DNA
catagccttaatccagcccg

SEQ ID NO: 54 - DNA, 24 bp, synthetic construct
  source 1..24: mol_type = other DNA
ccaagtgctgacaattaatacctg

SEQ ID NO: 55 - DNA, 25 bp, synthetic construct
  source 1..25: mol_type = other DNA
gacatagtttctgatggtacgtaga

SEQ ID NO: 56 - DNA, 24 bp, synthetic construct
  source 1..24: mol_type = other DNA
tcacgcctctctgaatctttgccg

SEQ ID NO: 57 - DNA, 19 bp, synthetic construct
  source 1..19: mol_type = other DNA
acatcgctcagacaccatg

SEQ ID NO: 58 - DNA, 22 bp, synthetic construct
  source 1..22: mol_type = other DNA
tgtagttgaggtcaatgaaggg

SEQ ID NO: 59 - DNA, 25 bp, synthetic construct
  source 1..25: mol_type = other DNA
aaggtcggagtcaacggatttggtc

SEQ ID NO: 60 - DNA, 21 bp, synthetic construct
  source 1..21: mol_type = other DNA
tacctgtgacaaccagtgttg

SEQ ID NO: 61 - DNA, 23 bp, synthetic construct
  source 1..23: mol_type = other DNA
acttacctgtaatgataagcttt

SEQ ID NO: 62 - DNA, 21 bp, synthetic construct
  source 1..21: mol_type = other DNA
ttaccaggcgaagttactatg

SEQ ID NO: 63 - DNA, 22 bp, synthetic construct
  source 1..22: mol_type = other DNA
acctggtgaagtcatacagttc

SEQ ID NO: 64 - DNA, 20 bp, synthetic construct
  source 1..20: mol_type = other DNA
cttccacttccactttgaaa

SEQ ID NO: 65 - DNA, 21 bp, synthetic construct
  source 1..21: mol_type = other DNA
actggtacctacaccaggagg

SEQ ID NO: 66 - DNA, 20 bp, synthetic construct
  source 1..20: mol_type = other DNA
agcatgggtaagacaagcaa

SEQ ID NO: 67 - protein, 512 aa, synthetic construct
QPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 68 - protein, 226 aa, synthetic construct
MPAMKIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRV

SEQ ID NO: 69 - protein, 280 aa, synthetic construct
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 70 - protein, 226 aa, synthetic construct; identical to SEQ ID NO: 68

SEQ ID NO: 71 - protein, 226 aa, synthetic construct; identical to SEQ ID NO: 68

SEQ ID NO: 72 - protein, 226 aa, synthetic construct; identical to SEQ ID NO: 68

SEQ ID NO: 73 - protein, 226 aa, synthetic construct; identical to SEQ ID NO: 68

SEQ ID NO: 74 - protein, 226 aa, synthetic construct; identical to SEQ ID NO: 68

SEQ ID NO: 75 - protein, 226 aa, synthetic construct; identical to SEQ ID NO: 68

SEQ ID NO: 76 - protein, 226 aa, synthetic construct; identical to SEQ ID NO: 68

SEQ ID NO: 77 - protein, 226 aa, synthetic construct; identical to SEQ ID NO: 68

SEQ ID NO: 78 - protein, 226 aa, synthetic construct; identical to SEQ ID NO: 68

SEQ ID NO: 79 - protein, 226 aa, synthetic construct; identical to SEQ ID NO: 68

SEQ ID NO: 80 - protein, 527 aa, synthetic construct
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 81 - protein, 280 aa, synthetic construct; identical to SEQ ID NO: 69

SEQ ID NO: 82 - protein, 828 aa, synthetic construct
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFDGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 83 - protein, 828 aa, synthetic construct; identical to SEQ ID NO: 82

SEQ ID NO: 84 - protein, 828 aa, synthetic construct; identical to SEQ ID NO: 82